(19) United States
(12) Patent Application Publication
(10) Pub. No.: US 2009/0118336 A1
(43) Pub. Date: May 7, 2009

(54) PYRAZOLE DERIVATIVES AND THEIR USE AS PI3K INHIBITORS

(76) Inventors: Laurent David, Lund (SE); Kevin Michael Foote, Macclesfield (GB); Annea Lisius, Lund (SE)

Correspondence Address: MORGAN LEWIS & BOCKIUS LLP, 1111 PENNSYLVANIA AVENUE NW, WASHINGTON, DC, 20004, US

(21) Appl. No.: 12/299,369

(22) PCT Filed: May 2, 2007
(86) PCT No.: PCT/GB2007/001619

§ 371 Date: Jan. 6, 2009

(30) Foreign Application Priority Data
May 3, 2006 (EP) 06300433.7

(51) Int. Cl. A61K 31/4439; C07D 401/14; C07D 417/14; C07D 401/04

(52) U.S. Cl. 514/341; 546/275.4; 514/342; 546/269.7

(57) ABSTRACT

The invention concerns pyrazole derivatives of Formula I

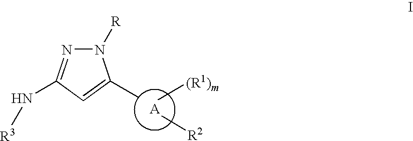

or pharmaceutically-acceptable salts thereof, wherein each of R, Ring A, m, R¹, R² and R³ has any of the meanings defined hereinbefore in the description; processes for their preparation, pharmaceutical compositions containing them and their use in therapy, for example in the treatment of disease mediated by a PI3K enzyme and/or a mTOR kinase.

The invention concerns certain novel pyrazole derivatives, or pharmaceutically-acceptable salts thereof, which possess anti-tumour activity and are accordingly useful in methods of treatment of the human or animal body. The invention also concerns processes for the manufacture of said pyrazole derivatives, pharmaceutical compositions containing them and their use in therapeutic methods, for example in the treatment of disease mediated by a PI3K enzyme and/or a mTOR kinase, for example in the manufacture of medicaments for use in the prevention or treatment of cancers in a warm-blooded animal such as man, including use in the production of an anti-proliferative effect and use in the prevention or treatment of solid tumour disease.

Many of the current treatment regimes for cell proliferation diseases such as cancer and psoriasis utilise compounds which inhibit DNA synthesis. Such compounds are toxic to cells generally but their toxic effect on rapidly dividing cells such as tumour cells can be beneficial. Alternative approaches to anti-tumour agents which act by mechanisms other than the inhibition of DNA synthesis have the potential to display enhanced selectivity of action.

In recent years it has been discovered that a cell may become cancerous by virtue of the transformation of a portion of its DNA into an oncogene, that is a gene which, on activation, leads to the formation of malignant tumour cells (Bradshaw, Mutagenesis, 1986, 1, 91). Several such oncogenes give rise to the production of peptides which are receptors for growth factors. Activation of the growth factor receptor complex subsequently leads to an increase in cell proliferation. It is known, for example, that several oncogenes encode tyrosine kinase enzymes and that certain growth factor receptors are also tyrosine kinase enzymes (Yarden et al., Ann. Rev. Biochem., 1988, 57, 443; Larsen et al., Ann. Reports in Med. Chem., 1989, Chpt. 13). The first group of tyrosine kinases to be identified arose from such viral oncogenes, for example pp60^{v-Src} tyrosine kinase (otherwise known as v-Src), and the corresponding tyrosine kinases in normal cells, for example pp60^{c-Src} tyrosine kinase (otherwise known as c-Src).

Receptor tyrosine kinases are important in the transmission of biochemical signals which initiate cell replication. They are large enzymes which span the cell membrane and possess an extracellular binding domain for growth factors such as epidermal growth factor (EGF) and an intracellular portion which functions as a kinase to phosphorylate tyrosine amino acids in proteins and hence to influence cell proliferation. Various classes of receptor tyrosine kinases are known (Wilks, Advances in Cancer Research, 1993, 60, 43-73) based on families of growth factors which bind to different receptor tyrosine kinases. The classification includes Class I receptor tyrosine kinases comprising the EGF family of receptor tyrosine kinases such as the EGF, TGFα, Neu and erbB receptors.

It is also known that certain tyrosine kinases belong to the class of non-receptor tyrosine kinases which are located intracellularly and are involved in the transmission of biochemical signals such as those that influence tumour cell motility, dissemination and invasiveness and subsequently metastatic tumour growth. Various classes of non-receptor tyrosine kinases are known including the Src family such as the Src, Lyn, Fyn and Yes tyrosine kinases.

It is also known that certain kinases belong to the class of serine/threonine kinases which are located intracellularly and downstream of tyrosine kinase activation and are involved in the transmission of biochemical signals such as those that influence tumour cell growth. Such serine/threonine signalling pathways include the Raf-MEK-ERK cascade and those downstream of the lipid kinase known as PI3K such as PDK-1, AKT and mTOR (Blume-Jensen and Hunter, Nature, 2001, 411, 355).

It is also known that the kinases that belong to the class of lipid kinases are located intracellularly and are also involved in the transmission of biochemical signals such as those that influence tumour cell growth and invasiveness. Various classes of lipid kinases are known including the phosphoinositide 3-kinase (abbreviated hereinafter to PI3K) family that is alternatively known as the phosphatidylinositol-3-kinase family.

It is now well understood that deregulation of oncogenes and tumour-suppressor genes contributes to the formation of malignant tumours, for example by way of increased cell proliferation or increased cell survival. It is also now known that signalling pathways mediated by the PI3K family have a central role in a number of cell processes including proliferation and survival, and deregulation of these pathways is a causative factor in a wide spectrum of human cancers and other diseases (Katso et al., Annual Rev. Cell Dev. Biol., 2001, 17: 615-617 and Foster et al., J. Cell Science, 2003, 116: 3037-3040).

The PI3K family of lipid kinases is a group of enzymes that phosphorylate the 3-position of the inositol ring of phosphatidylinositol (abbreviated hereinafter to PI). Three major groups of PI3K enzymes are known which are classified according to their physiological substrate specificity (Vanhaesebroeck et al., Trends in Biol. Sci., 1997, 22, 267). Class III PI3K enzymes phosphorylate PI alone. In contrast, Class II PI3K enzymes phosphorylate both PI and PI 4-phosphate [abbreviated hereinafter to PI(4)P]. Class I PI3K enzymes phosphorylate PI, PI(4)P and PI 4,5-bisphosphate [abbreviated hereinafter to PI(4,5)P2], although only PI(4,5)P2 is believed to be the physiological cellular substrate. Phosphorylation of PI(4,5)P2 produces the lipid second messenger PI 3,4,5-triphosphate [abbreviated hereinafter to PI(3,4,5)P3]. More distantly related members of this superfamily are Class IV kinases such as mTOR and DNA-dependent kinase that phosphorylate serine/threonine residues within protein substrates. The most studied and understood of these lipid kinases are the Class I PI3K enzymes.

Class I PI3K is a heterodimer consisting of a p110 catalytic subunit and a regulatory subunit, and the family is further divided into Class Ia and Class Ib enzymes on the basis of regulatory partners and mechanism of regulation. Class Ia enzymes consist of three distinct catalytic subunits (p110α, p110β and p110δ) that dimerise with five distinct regulatory subunits (p85α, p55α, p500α, p85β and p55γ), with all catalytic subunits being able to interact with all regulatory subunits to form a variety of heterodimers. Class Ia PI3K are generally activated in response to growth factor-stimulation of receptor tyrosine kinases, via interaction of the regulatory subunit SH2 domains with specific phospho-tyrosine residues of the activated receptor or adaptor proteins such as IRS-1. Both p110α and p110β are constitutively expressed in all cell types, whereas p110δ expression is more restricted to leukocyte populations and some epithelial cells. In contrast, the single Class Ib enzyme consists of a p110γ catalytic subunit that interacts with a p101 regulatory subunit. Furthermore, the Class Ib enzyme is activated in response to G-protein coupled receptor (GPCR) systems and its expression appears to be limited to leucocytes.

There is now considerable evidence indicating that Class Ia PI3K enzymes contribute to tumourigenesis in a wide variety of human cancers, either directly or indirectly (Vivanco and Sawyers, Nature Reviews Cancer, 2002, 29 489-501). For example, the p110α subunit is amplified in some tumours such as those of the ovary (Shayesteh et al., Nature Genetics, 1999, 21: 99-102) and cervix (Ma et al., Oncogene, 2000, 19: 2739-2744). More recently, activating mutations within the catalytic site of p110α have been associated with various other tumours such as those of the colorectal region and of the breast and lung (Samuels et al., Science, 2004, 304, 554). Tumour-related mutations in p85α have also been identified in cancers such as those of the ovary and colon (Philip et al., Cancer Research, 2001, 61, 7426-7429). In addition to direct effects, it is believed that activation of Class Ia PI3K contributes to tumourigenic events that occur upstream in signalling pathways, for example by way of ligand-dependent or ligand-independent activation of receptor tyrosine kinases, GPCR systems or integrins (Vara et al., Cancer Treatment Reviews, 2004, 30, 193-204). Examples of such upstream signalling pathways include over-expression of the receptor tyrosine kinase Erb2 in a variety of tumours leading to activation of PI3K-mediated pathways (Harari et al., Oncogene, 2000, 19, 6102-6114) and over-expression of the oncogene Ras (Kauffmann-Zeh et al., Nature, 1997, 385, 544-548). In addition, Class Ia PI3Ks may contribute indirectly to tumourigenesis caused by various downstream signalling events. For example, loss of the effect of the PTEN tumour-suppressor phosphatase that catalyses conversion of PI(3,4,5)P3 back to P1(4,5)P2 is associated with a very broad range of tumours via deregulation of PI3K-mediated production of PI(3,4,5)P3 (Simpson and Parsons, Exp. Cell Res., 2001, 264, 29-41). Furthermore, augmentation of the effects of other PI3K-mediated signalling events is believed to contribute to a variety of cancers, for example by activation of Akt (Nicholson and Anderson, Cellular Signalling, 2002, 14, 381-395).

In addition to a role in mediating proliferative and survival signalling in tumour cells, there is also good evidence that Class Ia PI3K enzymes will also contribute to tumourigenesis via its function in tumour-associated stromal cells. For example, PI3K signalling is known to play an important role in mediating angiogenic events in endothelial cells in response to pro-angiogenic factors such as VEGF (Abid et al., Arterioscler. Thromb. Vasc. Biol., 2004, 24, 294-300). As Class I PI3K enzymes are also involved in motility and migration (Sawyer, Expert Opinion Investig. Drugs, 2004, 13, 1-19), PI3K inhibitors should provide therapeutic benefit via inhibition of tumour cell invasion and metastasis.

In addition, Class I PI3K enzymes play an important role in the regulation of immune cells with PI3K activity contributing to pro-tumourigenic effects of inflammatory cells (Coussens and Werb, Nature, 2002, 420, 860-867).

These findings suggest that pharmacological inhibitors of Class I PI3K enzymes should be of therapeutic value for treatment of the various forms of the disease of cancer comprising solid tumours such as carcinomas and sarcomas and the leukaemias and lymphoid malignancies. In particular, inhibitors of Class I PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

PI3Kγ, the Class Ib PI3K, is activated by GPCRs, as was finally demonstrated in mice lacking the enzyme. Thus, neutrophils and macrophages derived from PI3Kγ-deficient animals failed to produce PI(3,4,5)P₃ in response to stimulation with various chemotactic substances (such as IL-8, C5a, fMLP and MIP-1α), whereas signalling through protein tyrosine kinase-coupled receptors to Class Ia PI3Ks was intact (Hirsch et al., Science, 2000, 287(5455), 1049-1053; Li et al., Science, 2002, 287(5455), 1046-1049; Sasaki et al., Science 2002, 287(5455), 1040-1046). Furthermore, PI(3,4,5)P₃-mediated phosphorylation of PKB was not initiated by these GPCR ligands in PI3Kγ-null cells. Taken together, the results demonstrated that, at least in resting haematopoietic cells, PI3Kγ is the sole PI3K isoform that is activated by GPCRs in vivo. When murine bone marrow-derived neutrophils and peritoneal macrophages from wild-type and PI3Kγ^{-/-} mice were tested in vitro, a reduced, but not completely abrogated, performance in chemotaxis and adherence assays was observed. However, this translated into a drastic impairment of IL-8 driven neutrophil infiltration into tissues (Hirsch et al., Science, 2000, 287(5455), 1049-1053. Recent data suggest that PI3Kγ is involved in the path-finding process rather than in the generation of mechanical force for motility, as random migration was not impaired in cells that lacked PI3Kγ (Hannigan et al., Proc. Nat. Acad. of Sciences of U.S.A., 2002, 99(6), 3603-8). Data linking PI3Kγ to respiratory disease pathology came with the demonstration that PI3Kγ has a central role in regulating endotoxin-induced lung infiltration and activation of neutrophils leading to acute lung injury (Yum et al., J. Immunology, 2001, 167(11), 6601-8). The fact that although PI3Kγ is highly expressed in leucocytes, its loss seems not to interfere with haematopoiesis, and the fact that PI3Kγ-null mice are viable and fertile further implicates this PI3K isoform as a potential drug target. Work with knockout mice also established that PI3Kγ is an essential amplifier of mast cell activation (Laffargue et al., Immunity, 2002, 16(3), 441-451).

Thus, in addition to tumourigenesis, there is evidence that Class I PI3K enzymes play a role in other diseases (Wymann et al., Trends in Pharmacological Science, 2003, 24, 366-376). Both Class Ia PI3K enzymes and the single Class Ib enzyme have important roles in cells of the immune system (Koyasu, Nature Immunology, 2003, 4, 313-319) and thus they are therapeutic targets for inflammatory and allergic indications. Inhibition of PI3K is also useful to treat cardiovascular disease via anti-inflammatory effects or directly by affecting cardiac myocytes (Prasad et al., Trends in Cardiovascular Medicine, 2003, 13, 206-212). Thus inhibitors of Class I PI3K enzymes are expected to be of value in the prevention and treatment of a wide variety of diseases in addition to cancer.

Generally, investigators have explored the physiological and pathological roles of the PI3K enzyme family using the PI3K inhibitors LY294002 and wortmannin. Although use of those compounds may suggest a role for PI3K in a cellular event, they are not sufficiently selective within the PI3K family to allow dissection of the individual roles of the family members. For this reason, more potent and selective pharmaceutical PI3K inhibitors would be useful to allow a more complete understanding of PI3K function and to provide useful therapeutic agents.

Accordingly, it would be desirable to provide further effective PI3K inhibitors for use in the treatment of cancer, inflammatory or obstructive airways diseases, immune or cardiovascular diseases.

International Patent Applications WO 03/072557, WO 2004/078754 and WO 2005/021519 describe 5-phenylthiazole derivatives as PI3K inhibitors. International Patent Application WO 2004/096797 describes certain 5-heteroaryl substituted thiazole derivatives as PI3K inhibitors. The heteroaryl group at the 5-position on the thiazole ring is a pyridin-4-yl group or a pyrimidin-4-yl group.

International Patent Application WO 2005/068444 describes certain 2-acylamino-5-thiazol-4-ylthiazole derivatives as PI3K inhibitors.

It has now been found that a series of pyrazole derivatives has inhibitory activity against the PI3K enzymes and against the Class IV kinase mTOR.

It is now well understood that deregulation of oncogenes and tumour-suppressor genes contributes to the formation of malignant tumours, for example by way of increased cell proliferation or increased cell survival. It is also now known that signalling pathways mediated by the PI3K/mTOR families have a central role in a number of cell processes including proliferation and survival, and deregulation of these pathways is a causative factor in a wide spectrum of human cancers and other diseases.

The mammalian target of the macrolide antibiotic Rapamycin (sirolimus) is the enzyme mTOR that belongs to the phosphatidylinositol (PI) kinase-related kinase (PIKK) family of protein kinases, which includes ATM, ATR, DNA-PK and hSMG-1. mTOR, like other PIKK family members, does not possess detectable lipid kinase activity, but instead functions as a serine/threonine kinase. Much of the knowledge of mTOR signalling is based upon the use of Rapamycin. Rapamycin first binds to the 12 kDa immunophilin FK506-binding protein (FKBP12) and this complex inhibits mTOR signalling (Tee and Blenis, Seminars in Cell and Developmental Biology, 2005, 16, 29-37). mTOR protein consists of a catalytic kinase domain, an FKBP12-Rapamycin binding (FRB) domain, a putative repressor domain near the C-terminus and up to 20 tandemly-repeated HEAT motifs at the N-terminus, as well as FRAP-ATM-TRRAP (FAT) and FAT C-terminus domain (Huang and Houghton, Current Opinion in Pharmacology, 2003, 3, 371-377).

mTOR kinase is a key regulator of cell growth and has been shown to regulate a wide range of cellular functions including translation, transcription, mRNA turnover, protein stability, actin cytoskeleton reorganisation and autophagy (Jacinto and Hall, Nature Reviews Molecular and Cell Biology, 2005, 4, 117-126). mTOR kinase integrates signals from growth factors (such as insulin or insulin-like growth factor) and nutrients (such as amino acids and glucose) to regulate cell growth. mTOR kinase is activated by growth factors through the PI3K-Akt pathway. The most well characterised function of mTOR kinase in mammalian cells is regulation of translation through two pathways, namely activation of ribosomal S6K1 to enhance translation of mRNAs that bear a 5′-terminal oligopyrimidine tract (TOP) and suppression of 4E-BP1 to allow CAP-dependent mRNA translation.

Generally, investigators have explored the physiological and pathological roles of mTOR using inhibition with Rapamycin and related Rapamycin analogues based on their specificity for mTOR as an intracellular target. However, recent data suggests that Rapamycin displays variable inhibitory actions on mTOR signalling functions and suggest that direct inhibition of the mTOR kinase domain may display substantially broader anti-cancer activities than that achieved by Rapamycin (Edinger et al., Cancer Research, 2003, 63, 8451-8460). For this reason, potent and selective inhibitors of mTOR kinase activity would be useful to allow a more complete understanding of mTOR kinase function and to provide useful therapeutic agents.

There is now considerable evidence indicating that the pathways upstream of mTOR are frequently activated in cancer (Vivanco and Sawyers, Nature Reviews Cancer, 2002, 2, 489-501; Bjornsti and Houghton, Nature Reviews Cancer, 2004, 4, 335-348; Inoki et al., Nature Genetics, 2005, 37, 19-24). For example, components of the PI3K pathway that are mutated in different human tumours include activating mutations of growth factor receptors and the amplification and/or overexpression of PI3K and Akt.

In addition there is evidence that endothelial cell proliferation may also be dependent upon mTOR signalling. Endothelial cell proliferation is stimulated by vascular endothelial cell growth factor (VEGF) activation of the PI3K-Akt-mTOR signalling pathway (Dancey, Expert Opinion on Investigational Drugs, 2005, 14, 313-328). Moreover, mTOR kinase signalling is believed to partially control VEGF synthesis through effects on the expression of hypoxia-inducible factor-1α (HIF-1α) (Hudson et al., Molecular and Cellular Biology, 2002, 22, 7004-7014). Therefore, tumour angiogenesis may depend on mTOR kinase signalling in two ways, through hypoxia-induced synthesis of VEGF by tumour and stromal cells, and through VEGF stimulation of endothelial proliferation and survival through PI3K-Akt-mTOR signalling.

These findings suggest that pharmacological inhibitors of mTOR kinase should be of therapeutic value for treatment of the various forms of the disease of cancer comprising solid tumours such as carcinomas and sarcomas and the leukaemias and lymphoid malignancies.

In addition to tumourigenesis, there is evidence that mTOR kinase plays a role in an array of hamartoma syndromes. Recent studies have shown that the tumour suppressor proteins such as TSC1, TSC2, PTEN and LKB1 tightly control mTOR kinase signalling. Loss of these tumour suppressor proteins leads to a range of hamartoma conditions as a result of elevated mTOR kinase signalling (Tee and Blenis, Seminars in Cell and Developmental Biology, 2005, 16, 29-37). Syndromes with an established molecular link to dysregulation of mTOR kinase include Peutz-Jeghers syndrome (PJS), Cowden disease, Bannayan-Riley-Ruvalcaba syndrome (BRRS), Proteus syndrome, Lhermitte-Duclos disease and TSC (Inoki et al., Nature Genetics, 2005, 37, 19-24). Patients with these syndromes characteristically develop benign hamartomatous tumours in multiple organs.

Recent studies have revealed a role for mTOR kinase in other diseases (Easton & Houghton, Expert Opinion on Therapeutic Targets, 2004, 8, 551-564). Rapamycin has been demonstrated to be a potent immunosuppressant by inhibiting antigen-induced proliferation of T cells, B cells and antibody production (Sehgal, Transplantation Proceedings, 2003, 35, 7S-14S) and thus mTOR kinase inhibitors may also be useful immunosuppressives. Inhibition of the kinase activity of mTOR may also be useful in the prevention of restenosis, that is the control of undesired proliferation of normal cells in the vasculature in response to the introduction of stents in the treatment of vasculature disease (Morice et al., New England Journal of Medicine, 2002, 346, 1773-1780). Furthermore, the Rapamycin analogue, everolimus, can reduce the severity and incidence of cardiac allograft vasculopathy (Eisen et al., New England Journal of Medicine, 2003, 349, 847-858). Elevated in TOR kinase activity has been associated with cardiac hypertrophy, which is of clinical importance as a major risk factor for heart failure and is a consequence of increased cellular size of cardiomyocytes (Tee & Blenis, Seminars in Cell and Developmental Biology, 2005, 16, 29-37). Thus mTOR kinase inhibitors are expected to be of value in the prevention and treatment of a wide variety of diseases in addition to cancer.

It has been found that certain pyrazole derivatives of the present invention have inhibitory activity against the mTOR PI kinase-related kinase family of enzymes as well as against PI3K enzymes.

We have found that surprisingly certain pyrazole derivatives possess potent anti-tumour activity, being useful in inhibiting the uncontrolled cellular proliferation which arises from malignant disease. Without wishing to imply that the compounds disclosed in the present invention possess pharmacological activity only by virtue of an effect on a single biological process, it is believed that the compounds provide an anti-tumour effect by way of inhibition of Class I PI3K enzymes, particularly by way of inhibition of the Class Ia PI3K enzymes and/or the Class Ib PI3K enzyme, more particularly by way of inhibition of the Class Ia PI3K enzymes.

The compounds of the present invention are also useful in inhibiting the uncontrolled cellular proliferation which arises from various non-malignant diseases such as inflammatory diseases (for example rheumatoid arthritis and inflammatory bowel disease), fibrotic diseases (for example hepatic cirrhosis and lung fibrosis), glomerulonephritis, multiple sclerosis, psoriasis, benign prostatic hypertrophy (BPH), hypersensitivity reactions of the skin, blood vessel diseases (for example atherosclerosis and restenosis), allergic asthma, insulin-dependent diabetes, diabetic retinopathy and diabetic nephropathy.

Generally, the compounds of the present invention possess potent inhibitory activity against Class I PI3K enzymes, particularly against Class Ia PI3K enzymes, whilst possessing less potent inhibitory activity against tyrosine kinase enzymes such as the receptor tyrosine kinases, for example EGF receptor tyrosine kinase and/or VEGF receptor tyrosine kinase, or against non-receptor tyrosine kinases such as Src. Furthermore, certain compounds of the present invention, possess substantially better potency against Class I PI3K enzymes, particularly against Class Ia PI3K enzymes, than against EGF receptor tyrosine kinase or VEGF receptor tyrosine kinase or Src non-receptor tyrosine kinase. Such compounds possess sufficient potency against Class I PI3K enzymes that they may be used in an amount sufficient to inhibit Class I PI3K enzymes, particularly to inhibit Class Ia PI3K enzymes, whilst demonstrating little activity against EGF receptor tyrosine kinase or VEGF receptor tyrosine kinase or Src non-receptor tyrosine kinase.

In accordance with the present invention, there is provided a pyrazole derivative of the Formula I

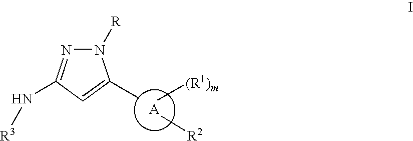

wherein:

the R group is hydrogen, (1-6C)alkyl or (3-8C)cycloalkyl, or the R group is a (1-3C)alkyl group that bears a substituent selected from cyano, hydroxy, amino, (1-6C)alkoxy, (1-6C)alkylthio, (1-6C)alkylsulphinyl, (1-6C)alkylsulphonyl, (1-6C)alkylamino, di-[(1-6C)alkyl]amino, phenoxy, benzyloxy, phenylthio, phenylsulphinyl and phenylsulphonyl,

and wherein any phenyl group within a R group optionally bears 1, 2 or 3 substituents, which may be the same or different, selected from halogeno, trifluoromethyl, cyano, hydroxy, amino, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino and di-[(1-6C)alkyl]amino;

Ring A is a 2-pyridyl, 3-pyridyl, 5-pyrimidinyl, 2-pyrazinyl or 4-pyridazinyl group;

m is 0, 1 or 2;

each R¹ group that is present, which may be the same or different, is selected from halogeno, trifluoromethyl, cyano, hydroxy, amino, (1-8C)alkyl, (2-8C)alkenyl, (2-8C)alkynyl and (1-6C)alkoxy;

the R² group is selected from halogeno, trifluoromethyl, cyano, hydroxy, amino, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X²-Q²

wherein X² is a direct bond or is selected from O, S, SO, SO₂, N(R⁵), CO, CH(OR⁵), CON(R⁵), N(R⁵)CO, N(R⁵)CON(R⁵), SO₂N(R⁵), N(R⁵)SO₂, C(R⁵)₂O, C(R⁵)₂S and C(R⁵)₂N(R⁵), wherein each R⁵ group is hydrogen, (1-8C)alkyl or (2-6C)alkanoyl, and Q² is aryl, aryl-(1-6C)alkyl, aryloxy-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH, CH₂ or CH₃ group within a R² group optionally bears on each said CH, CH₂ or CH₃ group one or more halogeno or (1-8C)alkyl substituents and/or a substituent selected from hydroxy, mercapto, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyloxy, (2-6C)alkanoylamino and N-(1-6C)alkyl-(2-6C)alkanoylamino, or from a group of the formula:

—X³-Q³

wherein X³ is a direct bond or is selected from O, S, SO, SO₂, N(R⁶) and CO, wherein R⁶ is hydrogen or (1-8C)alkyl, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R² group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:

—X⁴—R⁷

wherein X⁴ is a direct bond or is selected from O and N(R⁸), wherein R⁸ is hydrogen or (1-8C)alkyl, and R⁷ is halogeno-(1-6C)alkyl, hydroxy-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:

—X⁵-Q⁴

wherein X⁵ is a direct bond or is selected from O, CO and N(R⁹), wherein R⁹ is hydrogen or (1-8C)alkyl, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl,

and wherein any heterocyclyl group within the R² group optionally bears 1 or 2 oxo or thioxo substituents; and

the R³ group is selected from formyl, carboxy, carbamoyl, (1-6C)alkoxycarbonyl, N-(1-6C)alkylcarbamoyl, N,N-di-[(1-6C)alkyl]carbamoyl, (2-6C)alkanoyl, (3-8C)cycloalkylcarbonyl, N-(1-6C)alkylsulphamoyl and N,N-di-[(1-6C)alkyl]sulphamoyl, or from a group of the formula:

Q⁵-X⁶—

wherein X⁶ is selected from CO, N(R¹⁰)CO and N(R¹⁰)SO₂, wherein R¹⁰ is hydrogen or (1-8C)alkyl, and Q⁵ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH, CH₂ or CH₃ group within a R³ group optionally bears on each said CH, CH₂ or CH₃ group one or more halogeno or (1-8C)alkyl substituents and/or a substituent selected from hydroxy, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyloxy, (2-6C)alkanoylamino and N-(1-6C)alkyl-(2-6C)alkanoylamino,

and wherein any aryl, (3-8C)cycloalkyl, heteroaryl or heterocyclyl group within a R³ group optionally bears 1, 2 or 3 substituents, which may be the same or different, selected from halogeno, trifluoromethyl, cyano, nitro, trifluoromethoxy, hydroxy, amino, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyl, (2-6C)alkanoyloxy, (2-6C)alkanoylamino and N-(1-6C)alkyl-(2-6C)alkanoylamino,

and wherein any heterocyclyl group within a R³ group optionally bears 1 or 2 oxo or thioxo substituents;

or a pharmaceutically-acceptable salt thereof.

In this specification the generic term “(1-8C)alkyl” includes both straight-chain and branched-chain alkyl groups such as propyl, isopropyl and tert-butyl, and also (3-8C)cycloalkyl groups such as cyclopropyl, cyclobutyl, cyclopentyl, cyclohexyl and cycloheptyl, and also (3-6C)cycloalkyl-(1-2C)alkyl groups such as cyclopropylmethyl, 2-cyclopropylethyl, cyclobutylmethyl, 2-cyclobutylethyl, cyclopentylmethyl, 2-cyclopentylethyl, cyclohexylmethyl and 2-cyclohexylethyl. However references to individual alkyl groups such as “propyl” are specific for the straight-chain version only, references to individual branched-chain alkyl groups such as “isopropyl” are specific for the branched-chain version only and references to individual cycloalkyl groups such as “cyclopentyl” are specific for that 5-membered ring only. An analogous convention applies to other generic terms, for example (1-6C)alkoxy includes (3-6C)cycloalkyloxy groups and (3-5C)cycloalkyl-(1-2C)alkoxy groups, for example methoxy, ethoxy, propoxy, isopropoxy, cyclopropyloxy, cyclobutyloxy, cyclopentyloxy, cyclohexyloxy, cyclopropylmethoxy, 2-cyclopropylethoxy, cyclobutylmethoxy, 2-cyclobutylethoxy and cyclopentylmethoxy; (1-6C)alkylamino includes (3-6C)cycloalkylamino groups and (3-5C)cycloalkyl-(1-2C)alkylamino groups, for example methylamino, ethylamino, propylamino, cyclopropylamino, cyclobutylamino, cyclohexylamino, cyclopropylmethylamino, 2-cyclopropylethylamino, cyclobutylmethylamino, 2-cyclobutylethylamino and cyclopentylmethylamino; and di-[(1-6C)alkyl]amino includes di-[(3-6C)cycloalkyl]amino groups and di-[(3-5C)cycloalkyl-(1-2C)alkyl]amino groups, for example dimethylamino, diethylamino, dipropylamino, N-cyclopropyl-N-methylamino, N-cyclobutyl-N-methylamino, N-cyclohexyl-N-ethylamino, N-cyclopropylmethyl-N-methylamino, N-(2-cyclopropylethyl)-N-methylamino and N-cyclopentylmethyl-N-methylamino.

It is to be understood that, insofar as certain of the compounds of Formula I defined above may exist in optically active or racemic forms by virtue of one or more asymmetric carbon atoms, the invention includes in its definition any such optically active or racemic form which possesses the above-mentioned activity. The synthesis of optically active forms may be carried out by standard techniques of organic chemistry well known in the art, for example by synthesis from optically active starting materials or by resolution of a racemic form. Similarly, the above-mentioned activity may be evaluated using the standard laboratory techniques referred to hereinafter.

It is to be understood that certain compounds of Formula I defined above may exhibit the phenomenon of tautomerism. In particular, tautomerism may affect heterocyclic groups within the R² and R³ groups that bear 1 or 2 oxo or thioxo substituents. It is to be understood that the present invention includes in its definition any such tautomeric form, or a mixture thereof, which possesses the above-mentioned activity and is not to be limited merely to any one tautomeric form utilised within the formulae drawings or named in the Examples.

It is further to be understood that when Ring A is, for example, a 3-pyridyl group, the locant indicates the position that is linked to the 5-position on the pyrazole ring (counting from the N atom that carries the R group).

It is further to be understood that any R¹ group that is present on Ring A may be located at any available position on any of said 6-membered rings. When multiple R¹ groups are present, the R¹ groups may be the same or different, Conveniently, m is 0 and there is no R¹ group present on Ring A. Conveniently, there is a single R¹ group. Conveniently, the single R¹ group is located at the 2-, 3- or 4-position on Ring A (the locant being counted from the Ring A position that is linked to the 5-position on the pyrazole ring).

It is further to be understood that the R² group that is present on Ring A may be located at any available position on any of said 6-membered rings. Conveniently, the R¹ group is located at the 3- or 4-position on Ring A (the locant being counted from the Ring A position that is linked to the 5-position on the pyrazole ring). More conveniently, the R² group is located at the 3-position on Ring A.

Suitable values for the generic radicals referred to above include those set out below.

A suitable value for any one of the ‘Q’ groups (Q² to Q⁵) when it is aryl or for the aryl group within a ‘Q’ group is, for example, phenyl or naphthyl, preferably phenyl.

A suitable value for any one of the ‘Q’ groups (Q² to Q⁵) when it is (3-8C)cycloalkyl or for the (3-8C)cycloalkyl group within a ‘Q’ group is, for example, cyclopropyl, cyclobutyl, cyclopentyl, cyclohexyl, cycloheptyl, bicyclo[2.2.1]heptyl or cyclooctyl, or a benzo-fused (3-8C)cycloalkyl group such as indanyl or tetrahydronaphthyl.

A suitable value for any one of the ‘Q’ groups (Q² to Q⁵) when it is heteroaryl or for the heteroaryl group within a ‘Q’ group is, for example, an aromatic 5- or 6-membered monocyclic ring or a 9- or 10-membered bicyclic ring with up to five ring heteroatoms selected from oxygen, nitrogen and sulphur, for example furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, benzothienyl, benzoxazolyl, benzimidazolyl, benzothiazolyl, indazolyl, benzofurazanyl, quinolyl, isoquinolyl, quinazolinyl, quinoxalinyl, cinnolinyl or naphthyridinyl.

A suitable value for any one of the ‘Q’ groups (Q² to Q⁵) when it is heterocyclyl or for the heterocyclyl group within a ‘Q’ group is, for example, a non-aromatic saturated or partially saturated 3 to 10 membered monocyclic or bicyclic ring with up to five heteroatoms selected from oxygen, nitrogen and sulphur, for example oxiranyl, oxetanyl, tetrahydrofuranyl, tetrahydropyranyl, oxepanyl, tetrahydrothienyl, 1,1-dioxotetrahydrothienyl, tetrahydrothiopyranyl, 1,1-dioxotetrahydrothiopyranyl, azetidinyl, pyrrolinyl, pyrrolidinyl, imidazolinyl, imidazolidinyl, pyrazolinyl, pyrazolidinyl, morpholinyl, tetrahydro-1,4-thiazinyl, 1,1-dioxotetrahydro-1,4-thiazinyl, piperidinyl, homopiperidinyl, piperazinyl, homopiperazinyl, oxazolidine, thiazolidine, 2-azabicyclo[2.2.1]heptyl, quinuclidinyl, chromanyl, isochromanyl, indolinyl, isoindolinyl, dihydropyridinyl, tetrahydropyridinyl, dihydropyrimidinyl, tetrahydropyrimidinyl or tetrahydropyridazine, preferably tetrahydrofuranyl, tetrahydropyranyl, pyrrolidinyl, morpholinyl, piperidinyl or piperazinyl. A suitable value for such a group which bears 1 or 2 oxo or thioxo substituents is, for example, 2-oxopyrrolidinyl, 2-thioxopyrrolidinyl, 2-oxoimidazolidinyl, 2-thioxoimidazolidinyl, 2-oxooxazolidinyl, 2-oxothiazolidinyl, 2-oxopiperidinyl, 4-oxo-1,4-dihydropyridinyl, 2,5-dioxopyrrolidinyl, 2,5-dioxoimidazolidinyl or 2,6-dioxopiperidinyl.

A suitable value for a ‘Q’ group when it is heteroaryl-(1-6C)alkyl is, for example, heteroarylmethyl, 2-heteroarylethyl and 3-heteroarylpropyl. The invention comprises corresponding suitable values for ‘Q’ groups when, for example, rather than a heteroaryl-(1-6C)alkyl group, an aryl-(1-6C)alkyl, (3-8C)cycloalkyl-(1-6C)alkyl or heterocyclyl-(1-6C)alkyl group is present.

Suitable values for any of the ‘R’ groups (R, R¹ to R³ and R⁵ to R¹⁰), or for various groups within an R, R¹, R² or R³ group, or for various groups within any of the ‘Q’ groups (Q² to Q⁵) include:—
for (1-6C)alkyl: methyl, ethyl, propyl, isopropyl, butyl, isobutyl and tert-butyl;
for (3-8C)cycloalkyl: cyclopropyl, cyclobutyl, cyclopentyl and cyclohexyl;
for (1-3C)alkyl: methyl, ethyl, propyl and isopropyl;
for halogeno fluoro, chloro, bromo and iodo;
for (2-8C)alkenyl: vinyl, isopropenyl, allyl and but-2-enyl;
for (2-8C)alkynyl: ethynyl, 2-propynyl and but-2-ynyl;
for (1-6C)alkoxy: methoxy, ethoxy, propoxy, isopropoxy and butoxy;
for (2-6C)alkenyloxy: vinyloxy and allyloxy;
for (2-6C)alkynyloxy: ethynyloxy and 2-propynyloxy;
for (1-6C)alkylthio: methylthio, ethylthio and propylthio;
for (1-6C)alkylsulphinyl: methylsulphinyl and ethylsulphinyl;
for (1-6C)alkylsulphonyl: methylsulphonyl and ethylsulphonyl;
for (1-6C)alkylamino: methylamino, ethylamino, propylamino, isopropylamino and butylamino;
for di-[(1-6C)alkyl]amino: dimethylamino, diethylamino, N-ethyl-N-methylamino and diisopropylamino;
for (1-6C)alkoxycarbonyl: methoxycarbonyl, ethoxycarbonyl, propoxycarbonyl and tert-butoxycarbonyl;
for N-(1-6C)alkylcarbamoyl: N-methylcarbamoyl, N-ethylcarbamoyl and N-propylcarbamoyl;
for N,N-di-[(1-6C)alkyl]carbamoyl: N,N-dimethylcarbamoyl, N-ethyl-N-methylcarbamoyl and N,N-diethylcarbamoyl;
for (2-6C)alkanoyloxy: acetoxy and propionyloxy;
for (2-6C)alkanoylamino: acetamido and propionamido;
for N-(1-6C)alkyl-(2-6C)alkanoylamino: N-methylacetamido and N-methylpropionamido;
for N-(1-6C)alkylsulphamoyl: N-methylsulphamoyl and N-ethylsulphamoyl;
for N,N-di-[(1-6C)alkyl]sulphamoyl: N,N-dimethylsulphamoyl;
for (1-6C)alkanesulphonylamino: methanesulphonylamino and ethanesulphonylamino;
for N-(1-6C)alkyl-(1-6C)alkanesulphonylamino: N-methylmethanesulphonylamino and N-methylethanesulphonylamino;
for (1-8C)alkyl: methyl, ethyl, propyl, isopropyl, tert-butyl, cyclobutyl, cyclohexyl, cyclohexylmethyl and 2-cyclopropylethyl;
for (2-6C)alkanoyl: acetyl, propionyl and isobutyryl;
for halogeno-(1-6C)alkyl: chloromethyl, 2-fluoroethyl, 2-chloroethyl, 1-chloroethyl, 2,2-difluoroethyl, 2,2,2-trifluoroethyl, 3-fluoropropyl, 3-chloropropyl, 3,3-difluoropropyl and 3,3,3-trifluoropropyl;
for hydroxy-(1-6C)alkyl: hydroxymethyl, 2-hydroxyethyl, 1-hydroxyethyl and 3-hydroxypropyl;
for (1-6C)alkoxy-(1-6C)alkyl: methoxymethyl, ethoxymethyl, 1-methoxyethyl, 2-methoxyethyl, 2-ethoxyethyl and 3-methoxypropyl;
for (1-6C)alkylthio-(1-6C)alkyl: methylthiomethyl, ethylthiomethyl, 2-methylthioethyl, 1-methylthioethyl and 3-methylthiopropyl;
for (1-6C)alkylsulphinyl-(1-6C)alkyl: methylsulphinylmethyl, ethylsulphinylmethyl, 2-methylsulphinylethyl, 1-methylsulphinylethyl and 3-methylsulphinylpropyl;
for (1-6C)alkylsulphonyl-(1-6C)alkyl: methylsulphonylmethyl, ethylsulphonylmethyl, 2-methylsulphonylethyl, 1-methylsulphonylethyl and 3-methylsulphonylpropyl;
for cyano-(1-6C)alkyl: cyanomethyl, 2-cyanoethyl, 1-cyanoethyl and 3-cyanopropyl;
for amino-(1-6C)alkyl: aminomethyl, 2-aminoethyl, 1-aminoethyl, 3-aminopropyl, 1-aminopropyl and 5-aminopropyl;
for (1-6C)alkylamino-(1-6C)alkyl: methylaminomethyl, ethylaminomethyl, 1-methylaminoethyl, 2-methylaminoethyl, 2-ethylaminoethyl and 3-methylaminopropyl;
for di-[(1-6C)alkyl]amino-(1-6C)alkyl: dimethylaminomethyl, diethylaminomethyl, 1-dimethylaminoethyl, 2-dimethylaminoethyl and 3-dimethylaminopropyl;
for (2-6C)alkanoylamino-(1-6C)alkyl: acetamidomethyl, propionamidomethyl, 2-acetamidoethyl and 1-acetamidoethyl;
for N-(1-6C)alkyl-(2-6C)alkanoylamino-(1-6C)alkyl: N-methylacetamidomethyl, N-methylpropionamidomethyl, 2-(N-methylacetamido)ethyl and 1-(N-methylacetamido)ethyl; and
for (3-8C)cycloalkylcarbonyl: cyclopropylcarbonyl, cyclobutylcarbonyl, cyclopentylcarbonyl and cyclohexylcarbonyl.

When, as defined hereinbefore, R² is a group of the formula —X²-Q² and, for example, X² is a C(R⁵)₂O linking group, it is the carbon atom, not the oxygen atom, of the C(R⁵)₂O linking group which is attached to Ring A and the oxygen atom is linked to the Q² group.

Similarly, when, for example, R³ is a group of the formula Q⁵-X⁶— and, for example, X⁶ is a N(R¹⁰)CO linking group, it is the carbon atom, not the nitrogen atom, of the N(R¹⁰)CO linking group which is attached to the NH group at the 3-position on the pyrazole ring (counting from the N atom that carries the R group) and the nitrogen atom of the N(R¹⁰)CO linking group is attached to the Q⁵ group.

When, as defined hereinbefore, any CH, CH₂ or CH₃ group within a R² group or within a R³ group optionally bears a substituent as defined hereinbefore on each said CH, CH₂ or CH₃ group, it is to be understood that said CH and CH₂ groups form component parts of an acyclic R² or R³ group i.e. said CH and CH₂ groups do not form ring atoms within an aryl, (3-8C)cycloalkyl, heteroaryl or heterocyclyl ring.

When, as defined hereinbefore, any CH, CH₂ or CH₃ group within a R² group or within a R³ group optionally bears on each said CH, CH₂ or CH₃ group one or more halogeno or (1-8C)alkyl substituents, there is suitably 1 halogeno or (1-8C)alkyl substituent present on each said CH group, there are suitably 1 or 2 such substituents present on each said CH₂ group and there are suitably 1, 2 or 3 such substituents present on each said CH₃ group.

When, as defined hereinbefore, any CH, CH₂ or CH₃ group within a R² group or within a R³ group optionally bears on each said CH, CH₂ or CH₃ group a substituent as defined hereinbefore, suitable R² or R³ groups so formed include, for example, hydroxy-substituted (1-8C)alkyl groups such as hydroxymethyl, 1-hydroxyethyl and 2-hydroxyethyl, hydroxy-substituted (1-6C)alkoxy groups such as 2-hydroxypropoxy and 3-hydroxypropoxy, (1-6C)alkoxy-substituted (1-6C)alkoxy groups such as 2-methoxyethoxy and 3-ethoxypropoxy, hydroxy-substituted amino-(2-6C)alkoxy groups such as 3-amino-2-hydroxypropoxy, hydroxy-substituted (1-6C)alkylamino-(2-6C)alkoxy groups such as 2-hydroxy-3-methylaminopropoxy, hydroxy-substituted di-[(1-6C)alkyl]amino-(2-6C)alkoxy groups such as 3-dimethylamino-2-hydroxypropoxy, hydroxy-substituted amino-(2-6C)alkylamino groups such as 3-amino-2-hydroxypropylamino, hydroxy-substituted (1-6C)alkylamino-(2-6C)alkylamino groups such as 2-hydroxy-3-methylaminopropylamino and hydroxy-substituted di-[(1-6C)alkyl]amino-(2-6C)alkylamino groups such as 3-dimethylamino-2-hydroxypropylamino.

It is further to be understood that when, as defined hereinbefore, any CH, CH₂ or CH₃ group within a R² or R³ group optionally bears on each said CH, CH₂ or CH₃ group a substituent as defined hereinbefore, such an optional substituent may be present on a CH, CH₂ or CH₃ group within the hereinbefore defined substituents that may be present on an aryl, (3-8C)cycloalkyl, heteroaryl or heterocyclyl group within a R¹ or R³ group. For example, if R² includes an aryl or heteroaryl group that is substituted by a (1-8C)alkyl group, the (1-8C)alkyl group may be optionally substituted on a CH, CH₂ or CH₃ group therein by one of the hereinbefore defined substituents therefor. For example, if R² includes a heteroaryl group that is substituted by, for example, a (1-6C)alkylamino-(1-6C)alkyl group, the terminal CH₃ group of the (1-6C)alkylamino group may be further substituted by, for example, a (1-6C)alkylsulphonyl group or a (2-6C)alkanoyl group. For example, the R² group may be a heteroaryl group such as a thienyl group that is substituted by a N-(2-methylsulphonylethyl)aminomethyl group such that R² is, for example, a 5-[N-(2-methylsulphonylethyl)aminomethyl]thien-2-yl group. Further, for example, if R² includes a heterocyclyl group such as a piperidinyl or piperazinyl group that is substituted on a nitrogen atom thereof by, for example, a (2-6C)alkanoyl group, the terminal CH₃ group of the (2-6C)alkanoyl group may be further substituted by, for example, a di-[(1-6C)alkyl]amino group. For example, the R² group may be a N-(2-dimethylaminoacetyl)piperidin-4-yl group or a 4-(2-dimethylaminoacetyl)piperazin-1-yl group.

A suitable pharmaceutically-acceptable salt of a compound of the Formula I is, for example, an acid-addition salt of a compound of the Formula I, for example an acid-addition salt with an inorganic or organic acid such as hydrochloric, hydrobromic, sulphuric, trifluoroacetic, citric or maleic acid; or, for example, a salt of a compound of the Formula I which is sufficiently acidic, for example an alkali or alkaline earth metal salt such as a calcium or magnesium salt, or an ammonium salt, or a salt with an organic base such as methylamine, dimethylamine, trimethylamine, piperidine, morpholine or tris-(2-hydroxyethyl)amine. A further suitable pharmaceutically-acceptable salt of a compound of the Formula I is, for example, a salt formed within the human or animal body after administration of a compound of the Formula I.

It is further to be understood that a suitable pharmaceutically-acceptable solvate of a compound of the Formula I also forms an aspect of the present invention. A suitable pharmaceutically-acceptable solvate is, for example, a hydrate such as a hemi-hydrate, a mono-hydrate, a di-hydrate or a tri-hydrate or an alternative quantity thereof.

It is further to be understood that a suitable pharmaceutically-acceptable pro-drug of a compound of the Formula I also forms an aspect of the present invention. Accordingly, the compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the Formula I and in vivo cleavable amide derivatives that may be formed at a carboxy group or an amino group in a compound of the Formula I.

Accordingly, the present invention includes those compounds of the Formula I as defined hereinbefore when made available by organic synthesis and when made available within the human or animal body by way of cleavage of a pro-drug thereof. Accordingly, the present invention includes those compounds of the Formula I that are produced by organic synthetic means and also such compounds that are produced in the human or animal body by way of metabolism of a precursor compound, that is a compound of the Formula I may be a synthetically-produced compound or a metabolically-produced compound.

A suitable pharmaceutically-acceptable pro-drug of a compound of the Formula I is one that is based on reasonable medical judgement as being suitable for administration to the human or animal body without undesirable pharmacological activities and without undue toxicity.

Various forms of pro-drug have been described, for example in the following documents:—

a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);

### d) H. Bundgaard, Advanced Drug Delivery Reviews, 8, 1-38 (1992);

### e) H. Bundgaard, et al., Journal of Pharmaceutical Sciences, 77, 285 (1988);

f) N. Kakeya, et al., Chem. Pharm. Bull., 32, 692 (1984);

### g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

A suitable pharmaceutically-acceptable pro-drug of a compound of the Formula I that possesses a carboxy group is, for example, an in vivo cleavable ester thereof. An in vivo cleavable ester of a compound of the Formula I containing a carboxy group is, for example, a pharmaceutically-acceptable ester which is cleaved in the human or animal body to produce the parent acid. Suitable pharmaceutically-acceptable esters for carboxy include (1-6C)alkyl esters such as methyl, ethyl and tert-butyl, (1-6C)alkoxymethyl esters such as methoxymethyl esters, (1-6C)alkanoyloxymethyl esters such as pivaloyloxymethyl esters, 3-phthalidyl esters, (3-8C)cycloalkylcarbonyloxy-(1-6C)alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and (1-6C)alkoxycarbonyloxy-(1-6C)alkyl esters such as methoxycarbonyloxymethyl and 1-methoxycarbonyloxyethyl esters.

A suitable pharmaceutically-acceptable pro-drug of a compound of the Formula I that possesses a hydroxy group is, for example, an in vivo cleavable ester or ether thereof. An in vivo cleavable ester or ether of a compound of the Formula I containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include (1-10C)alkanoyl groups such as acetyl, benzoyl, phenylacetyl and substituted benzoyl and phenylacetyl groups, (1-10C)alkoxycarbonyl groups such as ethoxycarbonyl, N,N-[di-(1-4C)alkyl]carbamoyl, 2-dialkylaminoacetyl and 52-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl. Suitable pharmaceutically-acceptable ether forming groups for a hydroxy group include α-acyloxyalkyl groups such as acetoxymethyl and pivaloyloxymethyl groups.

A suitable pharmaceutically-acceptable pro-drug of a compound of the Formula I that possesses a carboxy group is, for example, an in vivo cleavable amide thereof, for example an amide formed with an amine such as ammonia, a (1-4C)alkylamine such as methylamine, a di-(1-4C)alkylamine such as dimethylamine, N-ethyl-N-methylamine or diethylamine, a (1-4C)alkoxy-(2-4C)alkylamine such as 2-methoxyethylamine, a phenyl-(1-4C)alkylamine such as benzylamine and amino acids such as glycine or an ester thereof.

A suitable pharmaceutically-acceptable pro-drug of a compound of the Formula I that possesses an amino group is, for example, an in vivo cleavable amide derivative thereof. Suitable pharmaceutically-acceptable amides from an amino group include, for example an amide formed with (1-10C)alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(1-4C)alkylpiperazin-1-ylmethyl.

The in vivo effects of a compound of the Formula I may be exerted in part by one or more metabolites that are formed within the human or animal body after administration of a compound of the Formula I. As stated hereinbefore, the in vivo effects of a compound of the Formula I may also be exerted by way of metabolism of a precursor compound (a pro-drug).

In a further aspect of the invention, there is provided a pyrazole derivative of the Formula II

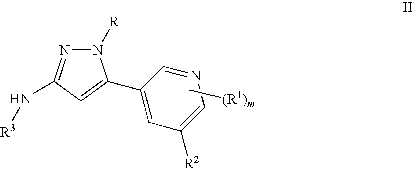

wherein each of R, m, R¹, R² and R³ has any of the meanings defined hereinbefore.

In a further aspect of the invention, there is provided a pyrazole derivative of the Formula II wherein R² is a (1-6C)alkylamino group or a group of the formula:

—NH-Q²

wherein Q² has any of the meanings defined hereinbefore and each of R, m, R¹ and R³ has any of the meanings defined hereinbefore.

In a further aspect of the invention, there is provided a pyrazole derivative of the Formula II wherein R² is a (1-6C)alkanesulphonylamino group or a group of the formula:

—NHSO₂-Q²

wherein Q² has any of the meanings defined hereinbefore and each of R, m, R¹ and R³ has any of the meanings defined hereinbefore.

In a further aspect of the invention, there is provided a pyrazole derivative of the Formula III

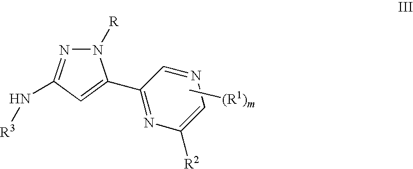

wherein each of R, m, R¹, R² and R³ has any of the meanings defined hereinbefore.

In a further aspect of the invention, there is provided a pyrazole derivative of the Formula IV

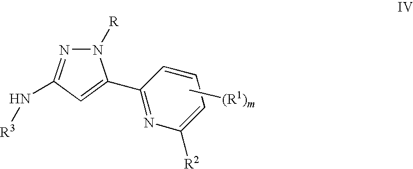

wherein each of R, m, R¹, R² and R³ has any of the meanings defined hereinbefore.

In a further aspect of the invention, there is provided a pyrazole derivative of the Formula V

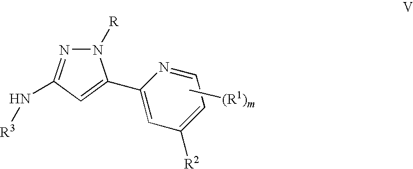

wherein each of R, m, R¹, R² and R³ has any of the meanings defined hereinbefore.

In a further aspect of the invention, there is provided a pyrazole derivative of the Formula VI

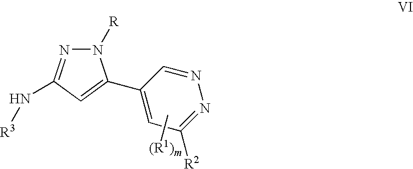

wherein each of R, m, R¹, R² and R³ has any of the meanings defined hereinbefore.

Particular novel compounds of the invention include, for example, pyrazole derivatives of the Formula I, or pharmaceutically-acceptable salts thereof, wherein, unless otherwise stated, each of R, Ring A, m, R¹, R² and R³ has any of the meanings defined hereinbefore or in paragraphs (a) to (hh) hereinafter. Particular novel compounds of the invention also include, for example, pyrazole derivatives of any of the Formulae II to VI, or pharmaceutically-acceptable salts thereof, wherein, unless otherwise stated, each of R, m, R¹, R² and R³ has any of the meanings defined hereinbefore or within appropriate paragraphs selected from paragraphs (a) to (hh) hereinafter:—

(a) R has any of the meanings defined hereinbefore other than hydrogen;
(b) the R group is (1-6C)alkyl (conveniently (1-3C)alkyl such as methyl, ethyl or propyl, particularly methyl);
(c) the R group is (3-8C)cycloalkyl (conveniently cyclopropyl);
(d) R is an ethyl group that bears a substituent selected from hydroxy, amino, (1-6C)alkoxy, (1-6C)alkylthio, (1-6C)alkylsulphinyl, (1-6C)alkylsulphonyl, (1-6C)alkylamino and di-[(1-6C)alkyl]amino;
(e) R is an ethyl group that bears a substituent selected from hydroxy, amino, methoxy, ethoxy, propoxy, methylsulphonyl, ethylsulphonyl, methylamino and dimethylamino;
(f) R is a 2-hydroxyethyl group;
(g) Ring A is a 2-pyridyl, 3-pyridyl or 2-pyrazinyl group;
(h) Ring A is a 3-pyridyl group;
(i) Ring A is a 5-pyrimidinyl group;
(j) Ring A is a 4-pyridazinyl group;
(k) m is 0;
(l) m is 1 or 2 and each R¹ group that is present, which may be the same or different, is selected from halogeno, trifluoromethyl, cyano, hydroxy, (1-6C)alkyl, (2-6C)alkenyl, (2-6C)alkynyl and (1-6C)alkoxy;
(m) m is 1 and the R¹ group is selected from halogeno, trifluoromethyl, cyano, hydroxy, (1-6C)alkyl, (2-6C)alkenyl, (2-6C)alkynyl and (1-6C)alkoxy;
(n) m is 1 and the R¹ group is selected from halogeno, (1-6C)alkyl and (1-6C)alkoxy;
(o) m is 1 and the R¹ group is selected from fluoro, chloro, bromo, methyl, ethyl and methoxy;
(p) m is 0 or m is 1 and the R¹ group is selected from fluoro, chloro, bromo, trifluoromethyl, cyano, methyl, ethyl, methoxy and ethoxy;
(q) m is 0 or m is 1 and the R¹ group is selected from fluoro, chloro, bromo, methyl, ethyl and methoxy;
(r) the R² group is selected from halogeno, trifluoromethyl, cyano, hydroxy, amino, (1-8C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)allyl-(1-6C)alkanesulphonylamino, or from a group of the formula:

—X²-Q²

wherein X² is selected from O, S, SO, SO₂, N(R⁵), CO, CON(R⁵), N(R⁵)CO, N(R⁵)CON(R⁵), SO₂N(R⁵) and N(R⁵)SO₂, wherein each R⁵ group is hydrogen, (1-8C)alkyl or (2-6C)alkanoyl, and Q² is aryl, aryl-(1-6C)alkyl, aryloxy-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH, CH₂ or CH₃ group within a R² group optionally bears on each said CH, CH₂ or CH₃ group one or more halogeno or (1-8C)alkyl substituents and/or a substituent selected from hydroxy, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoylamino and N-(1-6C)alkyl-(2-6C)alkanoylamino, or from a group of the formula:

—X³-Q³

is wherein X³ is a direct bond or is selected from O, S, SO, SO₂, N(R⁶) and CO, wherein R⁶ is hydrogen or (1-8C)alkyl, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R² group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (1-6C)alkoxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, N-(1-6C)alkylcarbamoyl, N,N-di-[(1-6C)alkyl]carbamoyl, (2-6C)alkanoylamino and N-(1-6C)alkyl-(2-6C)alkanoylamino, or from a group of the formula:

—X⁴—R⁷

wherein X⁴ is a direct bond or is selected from O and N(R⁸), wherein R⁸ is hydrogen or (1-8C)alkyl, and R⁷ is hydroxy-(1-6C)alkyl, (1-6C)alkoxy-(1-6C)alkyl, cyano-(1-6C)alkyl, amino-(1-6C)alkyl, (1-6C)alkylamino-(1-6C)alkyl and di-[(1-6C)alkyl]amino-(1-6C)alkyl, or from a group of the formula:

—X⁵-Q⁴

wherein X⁵ is a direct bond or is selected from O, CO and N(R⁹), wherein R⁹ is hydrogen or (1-8C)alkyl, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl,

and wherein any heterocyclyl group within the R² group optionally bears 1 or 2 oxo or thioxo substituents;

(s) the R² group is selected from (1-6C)alkoxy, (1-6C)alkylsulphonyl, (1-6C)alkylamino, di-[(1-6C)alkyl]amino, N-(1-6C)alkylcarbamoyl, (2-6C)alkanoylamino, (1-6C)alkanesulphonylamino and N-(1-6C)alkylsulphamoyl, or from a group of the formula:

—X²-Q²

wherein X² is selected from O, SO₂, NH, CONH, NHCO, SO₂NH and NHSO₂, and Q² is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH₂ or CH₃ group within a R² group optionally bears on each said CH₂ or CH₃ group one or more halogeno or (1-8C)alkyl substituents and/or a substituent selected from hydroxy, (1-6C)alkoxy, (1-6C)alkylamino and di-[(1-6C)alkyl]amino, or from a group of the formula:

—X³-Q³

wherein X³ is a direct bond or is selected from O and NH, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R¹ group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (1-6C)alkoxy, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino and N-(1-6C)alkyl-(2-6C)alkanoylamino, or from a group of the formula:

—X⁴—R⁷

wherein X⁴ is O and R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:

—X⁵-Q⁴

wherein X⁵ is a direct bond or O, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl; (t) the R² group is selected from (1-6C)alkylamino, di-[(1-6C)alkyl]amino, (2-6C)alkanoylamino and (1-6C)alkanesulphonylamino, or from a group of the formula:

—X²-Q²

wherein X² is selected from NH, NHCO and NHSO₂, and Q² is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH₂ or CH₃ group within a R² group optionally bears on each said CH₂ or CH₃ group one or more halogeno or (1-8C)alkyl substituents and/or a substituent selected from hydroxy, (1-6C)alkoxy, (1-6C)alkylamino and di-[(1-6C)alkyl]amino, or from a group of the formula:

—X³-Q³

wherein X³ is a direct bond or is selected from O and NH, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R¹ group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (1-6C)alkoxy, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino and N-(1-6C)alkyl-(2-6C)alkanoylamino, or from a group of the formula:

—X⁴—R⁷

wherein X⁴ is O and R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:

—X⁵-Q⁴

wherein X⁵ is a direct bond or O, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl;
(u) R² is a (1-6C)alkylamino group or a group of the formula:

—NH-Q²

wherein Q² is aryl-(1-6C)alkyl, (3-8C)cycloalkyl-(1-6C)alkyl, heteroaryl-(1-6C)alkyl or heterocyclyl-(1-6C)alkyl,

and wherein any aryl, (3-8C)cycloalkyl, heteroaryl or heterocyclyl group within a R² group optionally bears 1 or 2 substituents, which may be the same or different, selected from halogeno, trifluoromethyl, cyano, hydroxy, amino, (1-8C)alkyl, (1-6C)alkoxy, (1-6C)alkylsulphonyl, (1-6C)alkylamino, di-[(1-6C)alkyl]amino, (2-6C)alkanoyl and (2-6C)alkanoylamino, or from a group of the formula:

—O—R⁷

wherein R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:

—X⁵-Q⁴

wherein X¹ is a direct bond or O, and Q⁴ is aryl, aryl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, (1-8C)alkyl, (1-6C)alkoxy, (1-6C)alkylsulphonyl and (2-6C)alkanoyl;
(v) R² is a (1-6C)alkanesulphonylamino group or a group of the formula:

—NHSO₂-Q²

wherein Q² is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R² group optionally bears 1 or 2 substituents, which may be the same or different, selected from halogeno, trifluoromethyl, cyano, hydroxy, amino, carboxy, (1-8C)alkyl, (1-6C)alkoxy, (1-6C)alkylsulphonyl, (1-6C)alkylamino, di-[(1-6C)alkyl]amino, (1-6C)alkoxycarbonyl, (2-6C)alkanoyl, (2-6C)alkanoylamino and N-(1-6C)alkyl-(2-6C)alkanoylamino, or from a group of the formula:

—O—R⁷

wherein R⁷ is hydroxy-(1-6C)alkyl, (1-6C)alkoxy-(1-6C)alkyl, cyano-(1-6C)alkyl, (1-6C)alkylamino-(−6C)alkyl and di-[(1-6C)alkyl]amino-(1-6C)alkyl, or from a group of the formula:

—X⁵-Q⁴

wherein X¹ is a direct bond or O, and Q⁴ is aryl, aryl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, (1-8C)alkyl, (1-6C)alkoxy, (1-6C)alkylsulphonyl and (2-6C)alkanoyl;
(w) R² is methanesulphonylamino, ethanesulphonylamino, propanesulphonylamino, 2,2-difluoroethanesulphonylamino, 2,2,2-trifluoroethanesulphonylamino, 2-chloroethanesulphonylamino, 3-chloropropanesulphonylamino, 2-hydroxyethanesulphonylamino, 3-hydroxypropanesulphonylamino, 3-methylaminopropanesulphonylamino, 3-dimethylaminopropanesulphonylamino, 3-ethylaminopropanesulphonylamino, 3-diethylaminopropanesulphonylamino, 3-cyclopentylaminopropanesulphonylamino, 3-cyclohexylaminopropanesulphonylamino, 3-(cyclopentylmethylamino)propanesulphonylamino, 3-(cyclohexylmethylamino)propanesulphonylamino, 3-morpholinopropanesulphonylamino, 3-pyrrolidin-1-ylpropanesulphonylamino, 3-piperidinopropanesulphonylamino, 3-piperazin-1-ylpropanesulphonylamino, 3-(4-methylpiperazin-1-yl)propanesulphonylamino or 3-benzylaminopropanesulphonylamino, or R² is a group of the formula:

—N(R⁵)SO₂-Q²

wherein R⁵ is hydrogen, methyl, ethyl or acetyl, and Q² is phenyl, benzyl, cyclopropyl, cyclobutyl, cyclopentyl, cyclohexyl, cyclopropylmethyl, cyclobutylmethyl, pyrrolyl, furyl, thienyl, imidazolyl, pyrazolyl, oxazolyl, isoxazolyl, thiazolyl, isothiazolyl, oxadiazolyl, thiadiazolyl, triazolyl, pyridyl, pyrazinyl, pyrimidinyl or pyridazinyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, methyl, ethyl, methoxy, ethoxy, methylthio and methylsulphonyl;
(x) R² is methanesulphonylamino, ethanesulphonylamino or propanesulphonylamino, or a group of the formula:

—NHSO₂-Q²

wherein Q² is phenyl, benzyl, cyclopropyl, cyclopropylmethyl, 2-furyl, 3-furyl, 2-thienyl, 3-thienyl, 4-imidazolyl, 4-pyrazolyl, 5-oxazolyl, 4-isoxazolyl, 5-thiazolyl, 4-isothiazolyl or 3-pyridyl, each of which optionally bears 1 or 2 substituents, which may be the same or different, selected from fluoro, chloro, bromo, trifluoromethyl, cyano, hydroxy, amino, carboxy, methyl, ethyl, methoxy, ethoxy, methylsulphonyl, methylamino, dimethylamino, methoxycarbonyl, acetyl, acetamido and morpholino;
(y) R² is amino, methylamino, ethylamino, propylamino, dimethylamino, diethylamino, 2-hydroxyethylamino, 3-hydroxypropylamino, 3-methylaminopropylamino, 3-dimethylaminopropylamino, 3-ethylaminopropylamino or 3-diethylaminopropylamino, or R² is a group of the formula:

—N(R⁵)-Q²

wherein R⁵ is hydrogen, methyl or ethyl, and Q² is benzyl, pyrrolylmethyl, furylmethyl, thienylmethyl, imidazolylmethyl, pyrazolylmethyl, oxazolylmethyl, isoxazolylmethyl, thiazolylmethyl, isothiazolylmethyl, oxadiazolylmethyl, thiadiazolylmethyl, triazolylmethyl, pyridylmethyl, pyrazinylmethyl, pyrimidinylmethyl or pyridazinylmethyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, methyl, ethyl, methoxy, ethoxy, methylthio and methylsulphonyl;
(z) R¹ is a group of the formula:

—NH-Q²

wherein Q² is benzyl, 2-pyrrolylmethyl, 3-pyrrolylmethyl, 2-furylmethyl, 3-furylmethyl, 2-thienylmethyl, 3-thienylmethyl, 4-imidazolylmethyl, 4-pyrazolylmethyl, 5-oxazolylmethyl, 4-isoxazolylmethyl, 5-thiazolylmethyl, 4-isothiazolylmethyl, 1,2,3-triazol-4-ylmethyl and 3-pyridylmethyl, each of which optionally bears 1 or 2 substituents, which may be the same or different, selected from fluoro, chloro, bromo, trifluoromethyl, cyano, hydroxy, amino, carboxy, methyl, ethyl, methoxy, ethoxy, methylsulphonyl, methylamino, dimethylamino, methoxycarbonyl, acetyl and acetamido;
(aa) the R³ group is selected from formyl, carboxy, carbamoyl, (1-6C)alkoxycarbonyl, N-(1-6C)alkylcarbamoyl, N,N-di-[(1-6C)alkyl]carbamoyl, (2-6C)alkanoyl, (3-8C)cycloalkylcarbonyl, N-(1-6C)alkylsulphamoyl and N,N-di-[(1-6C)alkyl]sulphamoyl, or from a group of the formula:

Q⁵-X⁶—

wherein X⁶ is selected from CO, N(R¹⁰)CO and N(R¹⁰)SO₂, wherein R¹⁰ is hydrogen or (1-6C)alkyl, and Q⁵ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any CH, CH₂ or CH₃ group within a R³ group optionally bears on each said CH, CH₂ or CH₃ group one or more halogeno or (1-6C)alkyl substituents and/or a substituent selected from hydroxy, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyloxy, (2-6C)alkanoylamino and N-(1-6C)alkyl-(2-6C)alkanoylamino,

and wherein any aryl, (3-8C)cycloalkyl, heteroaryl or heterocyclyl group within a R³ group optionally bears 1, 2 or 3 substituents, which may be the same or different, selected from halogeno, trifluoromethyl, cyano, nitro, trifluoromethoxy, hydroxy, amino, (1-6C)alkyl, (2-6C)alkenyl, (2-6C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyl, (2-6C)alkanoyloxy, (2-6C)alkanoylamino and N-(1-6C)alkyl-(2-6C)alkanoylamino,

and wherein any heterocyclyl group within a R³ group optionally bears 1 or 2 oxo or thioxo substituents;

(bb) the R³ group is selected from carbamoyl, N-(1-6C)alkylcarbamoyl, N,N-di-[(1-6C)alkyl]carbamoyl and (2-6C)alkanoyl, or from a group of the formula:

Q⁵-X⁶—

wherein X⁶ is selected from CO and N(R¹⁰)CO, wherein R¹⁰ is hydrogen or (1-6C)alkyl, and Q⁵ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,

and wherein any aryl, (3-8C)cycloalkyl, heteroaryl or heterocyclyl group within a R³ group optionally bears 1, 2 or 3 substituents, which may be the same or different, selected from halogeno, trifluoromethyl, cyano, hydroxy, amino, (1-6C)alkyl, (1-6C)alkoxy, (1-6C)alkylsulphonyl, (1-6C)alkylamino, di-[(1-6C)alkyl]amino and (2-6C)alkanoyl,

and wherein any heterocyclyl group within a R³ group optionally bears 1 or 2 oxo or thioxo substituents;

(cc) the R³ group is selected from carbamoyl, N-(1-6C)alkylcarbamoyl and (2-6C)alkanoyl;
(dd) the R³ group is selected from carbamoyl, N-methylcarbamoyl, N-ethylcarbamoyl, N-isopropylcarbamoyl, acetyl and propionyl;
(ee) the R³ group is selected from acetyl and propionyl;
(ff) R³ is a group of the formula:

Q⁵-NHCO—

wherein Q⁵ is aryl-(1-6C)alkyl, (3-8C)cycloalkyl-(1-6C)alkyl or heteroaryl-(1-6C)alkyl,

and wherein any aryl, (3-8C)cycloalkyl or heteroaryl group within a R³ group optionally bears 1 or 2 substituents, which may be the same or different, selected from halogeno, trifluoromethyl, cyano, (1-6C)alkyl and (1-6C)alkoxy;

(gg) R³ is carbamoyl, N-methylcarbamoyl, N-ethylcarbamoyl, N-propylcarbamoyl, N-isopropylcarbamoyl, N-(2-hydroxyethyl)carbamoyl, N-(3-hydroxypropyl)carbamoyl, N-(2-methoxyethyl)carbamoyl, N-(3-methoxypropyl)carbamoyl, acetyl, propionyl, benzoyl, furylcarbonyl, thienylcarbonyl, pyridylcarbonyl, benzylcarbonyl, N-phenylcarbamoyl, N-benzylcarbamoyl, N-cyclopropylcarbamoyl, N-(furylmethyl)carbamoyl, N-(thienylmethyl)carbamoyl and N-(isoxazolylmethyl)carbamoyl, and each of the eleven last named substituents optionally bears 1 or 2 substituents, which may be the same or different, selected from fluoro, chloro, bromo, cyano, hydroxy, methyl, ethyl, methoxy and ethoxy; and
(hh) R³ is acetyl.

A particular compound of the invention is a pyrazole derivative of the Formula II

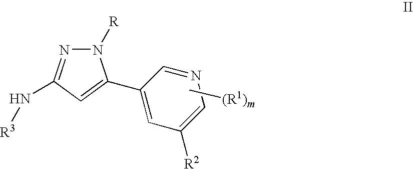

wherein R is methyl, ethyl or propyl;

m is 0 or m is 1 and the R¹ group is selected from fluoro, chloro, bromo, trifluoromethyl, cyano, methyl, ethyl, methoxy and ethoxy;

R² is methanesulphonylamino, ethanesulphonylamino, propanesulphonylamino, 2,2-difluoroethanesulphonylamino, 2,2,2-trifluoroethanesulphonylamino, 2-chloroethanesulphonylamino, 3-chloropropanesulphonylamino, 2-hydroxyethanesulphonylamino, 3-hydroxypropanesulphonylamino, 3-methylaminopropanesulphonylamino, 3-dimethylaminopropanesulphonylamino, 3-ethylaminopropanesulphonylamino, 3-diethylaminopropanesulphonylamino, 3-cyclopentylaminopropanesulphonylamino, 3-cyclohexylaminopropanesulphonylamino, 3-(cyclopentylmethylamino)propanesulphonylamino, 3-(cyclohexylmethylamino)propanesulphonylamino, 3-morpholinopropanesulphonylamino, 3-pyrrolidin-1-ylpropanesulphonylamino, 3-piperidinopropanesulphonylamino, 3-piperazin-1-ylpropanesulphonylamino, 3-(4-methylpiperazin-1-yl)propanesulphonylamino or 3-benzylaminopropanesulphonylamino, or R² is a group of the formula:

—N(R⁵)SO₂-Q²

wherein R⁵ is hydrogen, methyl, ethyl or acetyl, and Q² is phenyl, benzyl, cyclopropyl, cyclobutyl, cyclopentyl, cyclohexyl, cyclopropylmethyl, cyclobutylmethyl, pyrrolyl, furyl, thienyl, imidazolyl, pyrazolyl, oxazolyl, isoxazolyl, thiazolyl, isothiazolyl, oxadiazolyl, thiadiazolyl, triazolyl, pyridyl, pyrazinyl, pyrimidinyl or pyridazinyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, is methyl, ethyl, methoxy, ethoxy, methylthio and methylsulphonyl; and

R³ is carbamoyl, N-methylcarbamoyl, N-ethylcarbamoyl, N-propylcarbamoyl, N-isopropylcarbamoyl, N-(2-hydroxyethyl)carbamoyl, N-(3-hydroxypropyl)carbamoyl, N-(2-methoxyethyl)carbamoyl, N-(3-methoxypropyl)carbamoyl, acetyl, propionyl, benzoyl, furylcarbonyl, thienylcarbonyl, pyridylcarbonyl, benzylcarbonyl, N-phenylcarbamoyl, N-benzylcarbamoyl, N-cyclopropylcarbamoyl, N-(furylmethyl)carbamoyl, N-(thienylmethyl)carbamoyl and N-(isoxazolylmethyl)carbamoyl, and each of the eleven last named substituents optionally bears 1 or 2 substituents, which may be the same or different, selected from fluoro, chloro, bromo, cyano, hydroxy, methyl, ethyl, methoxy and ethoxy;

or a pharmaceutically-acceptable salt thereof.

A further particular compound of the invention is a pyrazole derivative of the Formula II wherein:—

R is methyl or ethyl;

m is 0 or m is 1 and the R¹ group is selected from fluoro, chloro, bromo, methyl, ethyl and methoxy;

R² is methanesulphonylamino, ethanesulphonylamino or propanesulphonylamino, or a group of the formula:

—NHSO₂-Q²

wherein Q² is phenyl, benzyl, cyclopropyl, cyclopropylmethyl, 2-furyl, 3-furyl, 2-thienyl, 3-thienyl, 4-imidazolyl, 4-pyrazolyl, 5-oxazolyl, 4-isoxazolyl, 5-thiazolyl, 4-isothiazolyl or 3-pyridyl, each of which optionally bears 1, 2 or 3 substituents, which may be the same or different, selected from fluoro, chloro, bromo, trifluoromethyl, cyano, hydroxy, amino, methyl, ethyl, methoxy, ethoxy, methylsulphonyl, methylamino, dimethylamino, acetyl and acetamido;

and R³ is acetyl;

or a pharmaceutically-acceptable salt thereof.

A further particular compound of the invention is a pyrazole derivative of the Formula II wherein:—

R is methyl;

m is 0 or m is 1 and the R¹ group is selected from chloro and methyl;

R² is methanesulphonylamino, or a group of the formula:

—NHSO₂-Q²

wherein Q² is phenyl, 5-thiazolyl or 4-pyrazolyl, each of which optionally bears 1, 2 or 3 substituents, which may be the same or different, selected from fluoro, chloro and methyl; and

R³ is acetyl;

or a pharmaceutically-acceptable salt thereof.

A further particular compound of the invention is a pyrazole derivative of the Formula II

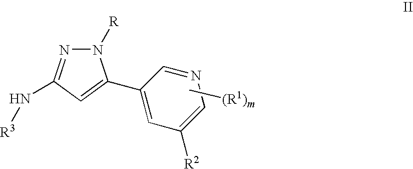

wherein:—

R is methyl, ethyl or propyl;

m is 0 or m is 1 and the R¹ group is selected from fluoro, chloro, bromo, trifluoromethyl, cyano, methyl, ethyl, methoxy and ethoxy;

R² is amino, methylamino, ethylamino, propylamino, dimethylamino, diethylamino, 2-hydroxyethylamino, 3-hydroxypropylamino, 3-methylaminopropylamino, 3-dimethylaminopropylamino, 3-ethylaminopropylamino or 3-diethylaminopropylamino, or R² is a group of the formula:

—N(R⁵)-Q²

wherein R⁵ is hydrogen, methyl or ethyl, and Q² is benzyl, pyrrolylmethyl, furylmethyl, thienylmethyl, imidazolylmethyl, pyrazolylmethyl, oxazolylmethyl, isoxazolylmethyl, thiazolylmethyl, isothiazolylmethyl, oxadiazolylmethyl, thiadiazolylmethyl, triazolylmethyl, pyridylmethyl, pyrazinylmethyl, pyrimidinylmethyl or pyridazinylmethyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, methyl, ethyl, methoxy, ethoxy, methylthio and methylsulphonyl; and

R³ is carbamoyl, N-methylcarbamoyl, N-ethylcarbamoyl, N-propylcarbamoyl, N-isopropylcarbamoyl, N-(2-hydroxyethyl)carbamoyl, N-(3-hydroxypropyl)carbamoyl, N-(2-methoxyethyl)carbamoyl, N-(3-methoxypropyl)carbamoyl, acetyl, propionyl, benzoyl, furylcarbonyl, thienylcarbonyl, pyridylcarbonyl, benzylcarbonyl, N-phenylcarbamoyl, N-benzylcarbamoyl, N-cyclopropylcarbamoyl, N-(furylmethyl)carbamoyl, N-(thienylmethyl)carbamoyl and N-(isoxazolylmethyl)carbamoyl, and each of the eleven last named substituents optionally bears 1 or 2 substituents, which may be the same or different, selected from fluoro, chloro, bromo, cyano, hydroxy, methyl, ethyl, methoxy and ethoxy;

or a pharmaceutically-acceptable salt thereof.

A further particular compound of the invention is a pyrazole derivative of the Formula II wherein:—

R is methyl or ethyl;

m is 0 or m is 1 and the R¹ group is selected from fluoro, chloro, bromo, methyl, ethyl and methoxy;

R² is a group of the formula:

—NH-Q²

wherein Q² is benzyl, 2-pyrrolylmethyl, 3-pyrrolylmethyl, 2-furylmethyl, 3-furylmethyl, 2-thienylmethyl, 3-thienylmethyl, 4-imidazolylmethyl, 4-pyrazolylmethyl, 5-oxazolylmethyl, 4-isoxazolylmethyl, 5-thiazolylmethyl, 4-isothiazolylmethyl, 1,2,3-triazol-4-ylmethyl and 3-pyridylmethyl, each of which optionally bears 1, 2 or 3 substituents, which may be the same or different, selected from fluoro, chloro, bromo, trifluoromethyl, cyano, hydroxy, amino, methyl, ethyl, methoxy, ethoxy, methylsulphonyl, methylamino, dimethylamino, acetyl and acetamido; and

R³ is acetyl;

or a pharmaceutically-acceptable salt thereof.

A further particular compound of the invention is a pyrazole derivative of the Formula II wherein:—

R is methyl;

m is 0 or m is 1 and the R¹ group is selected from chloro and methyl;

R² is a group of the formula: —NH-Q²

wherein Q² is 4-pyrazolylmethyl which optionally bears 1, 2 or 3 substituents, which may be the same or different, selected from fluoro, chloro and methyl; and

R³ is acetyl;

or a pharmaceutically-acceptable salt thereof.

A particular compound of the invention is, for example, a pyrazole derivative of the Formula I that is disclosed hereinafter amongst the Examples.

For example, a particular compound of the invention is a pyrazole derivative of the Formula I that is disclosed as Example 1, 2, 3 or 4, or a pharmaceutically-acceptable salt thereof.

A pyrazole derivative of the Formula I, or a pharmaceutically-acceptable salt thereof, may be prepared by any process known to be applicable to the preparation of chemically-related compounds. Such processes, when used to prepare a pyrazole derivative of the Formula I are provided as a further feature of the invention and are illustrated by the following representative Process Variants in which, unless otherwise stated, each of R, Ring A, m, R¹, R² and R³ have any of the meanings defined hereinbefore. Necessary starting materials may be obtained by standard procedures of organic chemistry. The preparation of such starting materials is described in conjunction with the following representative Process Variants and within the accompanying Examples. Alternatively, necessary starting materials are obtainable by analogous procedures to those illustrated which are within the ordinary skill of an organic chemist.

(a) The reaction, conveniently in the presence of a suitable catalyst, of a pyrazole of the Formula VII

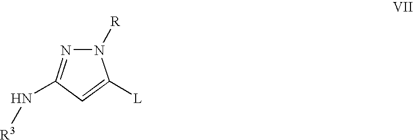

wherein L is a displaceable group and R and R³ have any of the meanings defined hereinbefore except that any functional group is protected if necessary, with an organoboron reagent of the Formula VIII

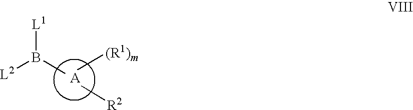

wherein each of L¹ and L², which may be the same or different, is a suitable ligand and Ring A, m, R¹ and R² have any of the meanings defined hereinbefore except that any functional group is protected if necessary, whereafter any protecting group that is present is removed.

A suitable displaceable group L is, for example, a halogeno, alkoxy, aryloxy or sulphonyloxy group, for example a chloro, bromo, iodo, methoxy, phenoxy, pentafluorophenoxy, methanesulphonyloxy or toluene-4-sulphonyloxy group. Conveniently, the displaceable group is an iodo group.

A suitable value for the ligands L¹ and L² which are present on the boron atom of the organoboron reagent include, for example, a hydroxy, (1-4C)alkoxy or (1-6C)alkyl ligand, for example a hydroxy, methoxy, ethoxy, propoxy, isopropoxy, butoxy, methyl, ethyl, propyl, isopropyl or butyl ligand. Alternatively the ligands L¹ and L² may be linked such that, together with the boron atom to which they are attached, they form a ring. For example, L¹ and L² together may define an oxy-(2-4C)alkylene-oxy group, for example an oxyethyleneoxy, oxytrimethyleneoxy group or —O—C(CH₃)₂C(CH₃)₂—O— group such that, together with the boron atom to which they are attached, they form a cyclic boronic acid ester group. Particularly suitable organoboron reagents include, for example, compounds wherein each of L¹ and L² is a hydroxy, a isopropoxy or an ethyl group or L¹ and L² together define a group of formula —O—C(CH₃)₂C(CH₃)₂—O—.

A suitable catalyst for the reaction includes, for example, a metallic catalyst such as a palladium(0), palladium(II), nickel(0) or nickel(II) catalyst, for example tetrakis(triphenylphosphine)palladium(0), palladium(II) chloride, palladium(II) bromide, bis(triphenylphosphine)palladium(II) chloride, tetrakis(triphenylphosphine)nickel(0), nickel(II) chloride, nickel(II) bromide, bis(triphenylphosphine)nickel(II) chloride or [1,1′-bis(diphenylphosphino)ferrocene]dichloropalladium(II). In addition, a free radical initiator may conveniently be added, for example an azo compound such as azo(bisisobutyronitrile). Conveniently, the reaction may be carried out in the presence of a suitable base such as an alkali or alkaline earth metal carbonate or hydroxide, for example sodium bicarbonate, sodium carbonate, potassium bicarbonate, potassium carbonate, calcium carbonate, caesium carbonate, sodium hydroxide or potassium hydroxide, or, for example, an alkali metal alkoxide, for example sodium tert-butoxide, or, for example, an alkali metal amide, for example sodium hexamethyldisilazane, or, for example, an alkali metal hydride, for example sodium hydride.

The reaction is conveniently carried out in the presence of a suitable inert solvent or diluent, for example an ether such as tetrahydrofuran, 1,4-dioxan or 1,2-dimethoxyethane, an aromatic solvent such as benzene, toluene or xylene, or an alcohol such as methanol or ethanol, and the reaction is conveniently carried out at a temperature in the range, for example, 10 to 250° C., preferably in the range 40 to 120° C.

Heteroaryl-boron reagents of the Formula VIII may be obtained by standard procedures of organic chemistry which are within the ordinary skill of an organic chemist. For example, a heteroaryl-metal reagent where the metal is, for example, lithium or the magnesium halide portion of a Grignard reagent, may be reacted with an organoboron compound of the formula L-B(L¹)(L²) wherein L is a displaceable group as defined hereinbefore. Preferably the compound of the formula L-B(L¹)(L²) is, for example, boric acid or a tri-(1-4C)alkyl borate such as tri-isopropyl borate.

Alternatively, for example, a heteroaryl-boron reagent of the Formula VIII may be replaced with an organometallic compound of the formula heteroaryl-M wherein M is a metal atom or a metallic group (that is a metal atom bearing suitable ligands). Suitable values for the metal atom include, for example, lithium and copper. Suitable values for the metallic group include, for example, groups which contain a tin, silicon, zirconium, aluminium, magnesium, mercury or zinc atom. Suitable ligands within such a metallic group include, for example, hydroxy groups, (1-6C)alkyl groups such as methyl, ethyl, propyl, isopropyl and butyl groups, halogeno groups such as chloro, bromo and iodo groups, and (1-6C)alkoxy groups such as methoxy, ethoxy, propoxy, isopropoxy and butoxy groups. A particular organometallic compound of the formula heteroaryl-M is, for example, an organotin compound such as a compound of the formula heteroaryl-SnBu₃, an organosilicon compound such as a compound of the formula heteroaryl-Si(Me)F₂, an organozirconium compound such as a compound of the formula heteroaryl-ZrCl₃, an organoaluminum compound such as a compound of the formula heteroaryl-AlEt₂, an organomagnesium compound such as a compound of the formula heteroaryl-MgBr, an organomercury compound such as a compound of the formula heteroaryl-HgBr, or an organozinc compound such as a compound of the formula heteroaryl-ZnBr.

Protecting groups may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Specific examples of protecting groups are given below for the sake of convenience, in which “lower”, as in, for example, lower alkyl, signifies that the group to which it is applied preferably has 1-4 carbon atoms. It will be understood that these examples are not exhaustive. Where specific examples of methods for the removal of protecting groups are given below these are similarly not exhaustive. The use of protecting groups and methods of deprotection not specifically mentioned are, of course, within the scope of the invention.

A carboxy protecting group may be the residue of an ester-forming aliphatic or arylaliphatic alcohol or of an ester-forming silanol (the said alcohol or silanol preferably containing 1-20 carbon atoms). Examples of carboxy protecting groups include straight or branched chain (1-12C)alkyl groups (for example isopropyl, and tert-butyl); lower alkoxy-lower alkyl groups (for example methoxymethyl, ethoxymethyl and isobutoxymethyl); lower acyloxy-lower alkyl groups, (for example acetoxymethyl, propionyloxymethyl, butyryloxymethyl and pivaloyloxymethyl); lower alkoxycarbonyloxyl-lower alkyl groups (for example 1-methoxycarbonyloxyethyl and 1-ethoxycarbonyloxyethyl); aryl-lower alkyl groups (for example benzyl, 4-methoxybenzyl, 2-nitrobenzyl, 4-nitrobenzyl, benzhydryl and phthalidyl); tri(lower alkyl)silyl groups (for example trimethylsilyl and tert-butyldimethylsilyl); tri(lower alkyl)silyl-lower alkyl groups (for example trimethylsilylethyl); and (2-6C)alkenyl groups (for example allyl). Methods particularly appropriate for the removal of carboxyl protecting groups include for example acid-, base-, metal- or enzymically-catalysed cleavage.

Examples of hydroxy protecting groups include lower alkyl groups (for example tert-butyl), lower alkenyl groups (for example allyl); lower alkanoyl groups (for example acetyl); lower alkoxycarbonyl groups (for example tert-butoxycarbonyl); lower alkenyloxycarbonyl groups (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); tri(lower alkyl)silyl (for example trimethylsilyl and tert-butyldimethylsilyl) and aryl-lower alkyl (for example benzyl) groups.

Examples of amino protecting groups include formyl, aryl-lower alkyl groups (for example benzyl and substituted benzyl, 4-methoxybenzyl, 2-nitrobenzyl and 2,4-dimethoxybenzyl, and triphenylmethyl); di-4-anisylmethyl and furylmethyl groups; lower alkoxycarbonyl (for example tert-butoxycarbonyl); lower alkenyloxycarbonyl (for example allyloxycarbonyl); aryl-lower alkoxycarbonyl groups (for example benzyloxycarbonyl, 4-methoxybenzyloxycarbonyl, 2-nitrobenzyloxycarbonyl and 4-nitrobenzyloxycarbonyl); trialkylsilyl (for example trimethylsilyl and tert-butyldimethylsilyl); alkylidene (for example methylidene) and benzylidene and substituted benzylidene groups.

Methods appropriate for removal of hydroxy and amino protecting groups include, for example, acid-, base-, metal- or enzymically-catalysed hydrolysis for groups such as 2-nitrobenzyloxycarbonyl, hydrogenation for groups such as benzyl and photolytically for groups such as 2-nitrobenzyloxycarbonyl.

The reader is referred to Advanced Organic Chemistry, 4th Edition, by J. March, published by John Wiley & Sons 1992, for general guidance on reaction conditions and reagents and to Protective Groups in Organic Synthesis, 2^{nd} Edition, by T. Green et al., also published by John Wiley & Son, for general guidance on protecting groups.

Pyrazole starting materials of the Formula VII may be obtained by conventional procedures such as those disclosed in the Examples that are set out hereinafter.

(b) The reaction, conveniently in the presence of a transition metal catalyst and conveniently in the presence of a suitable base, of a compound of the Formula IX

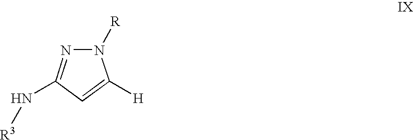

wherein R and R³ have any of the meanings defined hereinbefore except that any functional group is protected if necessary, with a compound of the Formula X

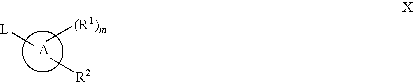

wherein L is a displaceable group as defined hereinbefore and Ring A, m, R¹ and R² have any of the meanings defined hereinbefore except that any functional group is protected if necessary, whereafter any protecting group that is present is removed.

A suitable transition metal catalyst for the reaction is, for example, a catalyst such as a palladium(0), palladium(II), nickel(0) or nickel(II) catalyst, for example tetrakis(triphenylphosphine)palladium(0), palladium(II) chloride, palladium(II) bromide, bis(triphenylphosphine)palladium(II) chloride, tris(dibenzylideneacetone)dipalladium(0) tetrakis(triphenylphosphine)nickel(0), nickel(II) chloride, nickel(II) bromide or bis(triphenylphosphine)nickel(II) chloride. Conveniently, the transition metal catalyst is a palladium catalyst, for example palladium(II) acetate.

Conveniently, a phosphine ligand for the transition metal is present, for example triphenylphosphine, tributylphosphine or 4,5-bis(diphenylphosphino)-9,9-dimethylxanthene. More conveniently, the phosphine ligand is tri-tert-butylphosphine.

A suitable base for the reaction is an alkali or alkaline earth metal carbonate or hydroxide, for example sodium bicarbonate, sodium carbonate, potassium bicarbonate, potassium carbonate, calcium carbonate, caesium carbonate, sodium hydroxide or potassium hydroxide. Conveniently, the reaction is carried out in the presence of caesium fluoride.

Conveniently, the process may be carried out in an organic solvent such as DMSO and the reaction temperature may be from about 60° C. to 200° C., conveniently at about 130° C. to 150° C.

Pyrazole starting materials of the Formula IX may be obtained by conventional procedures such as those disclosed in the scientific literature or within the Examples that are set out hereinafter. Likewise, compounds of the Formula X may be obtained by conventional procedures such as those disclosed in the scientific literature or within the Examples that are set out hereinafter.

(c) For the production of those compounds of the Formula I wherein R² is a (1-6C)alkanesulphonylamino group, the reaction, conveniently in the presence of a suitable base, of a compound of the Formula XI

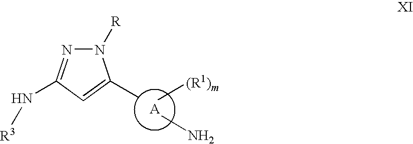

wherein R, Ring A, m, R¹, and R³ have any of the meanings defined hereinbefore except that any functional group is protected if necessary, with a (1-6C)alkanesulphonic acid, or a reactive derivative thereof, whereafter any protecting group that is present is removed by conventional means.

A suitable base for this alkanesulphonylation reaction is, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide, or, for example, an alkali metal amide, for example sodium hexamethyldisilazane, or, for example, an alkali metal hydride, for example sodium hydride.

A suitable reactive derivative of a (1-6C)alkanesulphonic acid is, for example, an alkanesulphonyl halide, for example an alkanesulphonyl chloride formed by the reaction of the sulphonic acid with an inorganic acid chloride, for example thionyl chloride or the product of the reaction of the sulphonic acid with a carbodiimide such as dicyclohexylcarbodiimide.

The reaction is conveniently carried out in the presence of a suitable inert solvent or diluent, for example an alcohol or ester such as methanol, ethanol, isopropanol or ethyl acetate, a halogenated solvent such as methylene chloride, chloroform or carbon tetrachloride, an ether such as tetrahydrofuran or 1,4-dioxan, an aromatic solvent such as toluene. Conveniently, the reaction is conveniently carried out in the presence of a dipolar aprotic solvent such as N,N-dimethylformamide, N,N-dimethylacetamide, N-methylpyrrolidin-2-one or dimethylsulphoxide. The reaction is conveniently carried out at a temperature in the range, for example, 0 to 120° C., preferably at or near ambient temperature.

Pyrazole starting materials of the Formula XI may be obtained conventionally, for example by way of Process Variants (a) or (b) as described hereinbefore and/or using procedures such as those disclosed within the Examples that are set out hereinafter.

(d) For the production of those compounds of the Formula I wherein R² is a group of the formula:—

—X²-Q²

wherein X² is a N(R⁵)SO₂ group and Q² has any of the meanings defined hereinbefore, the reaction, conveniently in the presence of a suitable base as defined hereinbefore, of a compound of the Formula XII

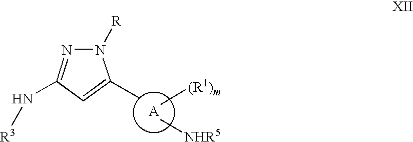

wherein R, Ring A, m, R¹, R³ and R⁵ have any of the meanings defined hereinbefore except that any functional group is protected if necessary, with a sulphonic acid of the formula:—

HO—SO₂-Q²

or a reactive derivative thereof, wherein Q² has any of the meanings defined hereinbefore except that any functional group is protected if necessary, whereafter any protecting group that is present is removed.

A suitable reactive derivative of a sulphonic acid of the formula:—

HO—SO₂-Q²

is, for example, a sulphonyl halide, for example a sulphonyl chloride formed by the reaction of the sulphonic acid with an inorganic acid chloride, for example thionyl chloride or the product of the reaction of the sulphonic acid with a carbodiimide such as dicyclohexylcarbodiimide.

The reaction is conveniently carried out in the presence of a suitable inert solvent or diluent as defined hereinbefore. The reaction is conveniently carried out at a temperature in the range, for example, 0 to 120° C., preferably at or near ambient temperature.

Pyrazole starting materials of the Formula XII may be obtained conventionally, for example by way of Process Variants (a) or (b) as described hereinbefore and/or using procedures such as those disclosed within the Examples that are set out hereinafter.

(e) For the production of those compounds of the Formula I wherein R² is a group of the formula:—

—X²-Q²

wherein X² is a SO₂N(R⁵) group and Q² has any of the meanings defined hereinbefore, the reaction, conveniently in the presence of a suitable base as defined hereinbefore, of a sulphonic acid of the Formula XIII

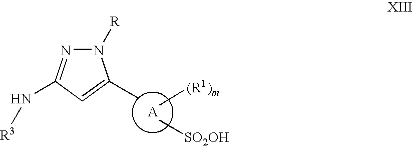

or a reactive derivative thereof as defined hereinbefore, wherein R, Ring A, m, R¹ and R³ have any of the meanings defined hereinbefore except that any functional group is protected if necessary, with an amine of the formula:—

R⁵NH-Q²

wherein R⁵ and Q² have any of the meanings defined hereinbefore except that any functional group is protected if necessary, whereafter any protecting group that is present is removed.

The reaction is conveniently carried out in the presence of a suitable inert solvent or diluent as defined hereinbefore. The reaction is conveniently carried out at a temperature in the range, for example, 0 to 120° C., preferably at or near ambient temperature.

Pyrazole starting materials of the Formula XIII may be obtained conventionally, for example by way of Process Variants (a) or (b) as described hereinbefore and/or using procedures that are analogous to those disclosed within the Examples that are set out hereinafter.

(f) For the production of those compounds of the Formula I wherein R² is a (2-6C)alkanoylamino group, the reaction, conveniently in the presence of a suitable base as defined hereinbefore, of a compound of the Formula XI

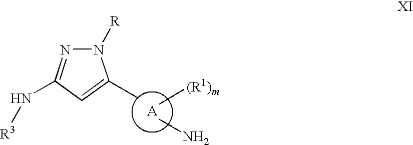

wherein R, Ring A, m, R, and R³ have any of the meanings defined hereinbefore except that any functional group is protected if necessary, with a (2-6C)alkanoic acid, or a reactive derivative thereof, whereafter any protecting group that is present is removed.

A suitable reactive derivative of a (2-6C)alkanoic acid is, for example, an acyl halide, for example an acyl chloride formed by the reaction of the acid with an inorganic acid chloride, for example thionyl chloride; a mixed anhydride, for example an anhydride formed by the reaction of the acid with a chloroformate such as isobutyl chloroformate; an active ester, for example an ester formed by the reaction of the acid with a phenol such as pentafluorophenol, with an ester such as pentafluorophenyl trifluoroacetate or with an alcohol such as methanol, ethanol, isopropanol, butanol or N-hydroxybenzotriazole; an acyl azide, for example an azide formed by the reaction of the acid with an azide such as diphenylphosphoryl azide; an acyl cyanide, for example a cyanide formed by the reaction of an acid with a cyanide such as diethylphosphoryl cyanide; or the product of the reaction of the acid with a carbodiimide such as dicyclohexylcarbodiimide or with a uronium compound such as 2-(7-azabenzotriazol-1-yl)-1,1,3,3-tetramethyluronium hexafluorophosphate(V).

The reaction is conveniently carried out in the presence of a suitable inert solvent or diluent as defined hereinbefore. The reaction is conveniently carried out at a temperature in the range, for example, 0 to 120° C., preferably at or near ambient temperature.

(g) For the production of those compounds of the Formula I wherein R² is a group of the formula:—

—X²-Q²

wherein X² is a N(R⁵)CO group and Q² has any of the meanings defined hereinbefore, the reaction, conveniently in the presence of a suitable base as defined hereinbefore, of a compound of the Formula XII

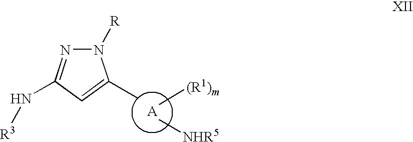

wherein R, Ring A, m, R¹, R³ and R⁵ have any of the meanings defined hereinbefore except that any functional group is protected if necessary, with a carboxylic acid of the formula

HO₂C-Q²

or a reactive derivative thereof, wherein Q² has any of the meanings defined hereinbefore except that any functional group is protected if necessary, whereafter any protecting group that is present is removed.

A suitable reactive derivative of a carboxylic acid of the formula

HO₂C-Q²

is, for example, an acyl chloride formed by the reaction of the acid with an inorganic acid chloride, for example thionyl chloride; or the product of the reaction of the acid with a carbodiimide such as dicyclohexylcarbodiimide or with a uronium compound such as 2-(7-azabenzotriazol-1-yl)-1,1,3,3-tetramethyluronium hexafluorophosphate(V).

The reaction is conveniently carried out in the presence of a suitable inert solvent or diluent as defined hereinbefore. The reaction is conveniently carried out at a temperature in the range, for example, 0 to 120° C., preferably at or near ambient temperature.

(h) For the production of those compounds of the Formula I wherein R² is a group of the formula:—

—X²-Q²

wherein X² is a CON(R⁵) group and Q² has any of the meanings defined hereinbefore, the reaction, conveniently in the presence of a suitable base as defined hereinbefore, of a carboxylic acid of the Formula XIV

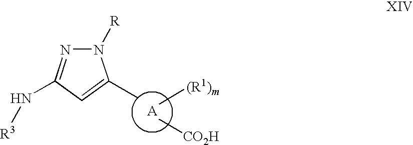

or a reactive derivative thereof as defined hereinbefore, wherein R, Ring A, m, R¹ and R³ have any of the meanings defined hereinbefore except that any functional group is protected if necessary, with an amine of the formula:—

R⁵NH-Q²

wherein R⁵ and Q² have any of the meanings defined hereinbefore except that any functional group is protected if necessary, whereafter any protecting group that is present is removed.

The reaction is conveniently carried out in the presence of a suitable inert solvent or diluent as defined hereinbefore. The reaction is conveniently carried out at a temperature in the range, for example, 0 to 120° C., preferably at or near ambient temperature.

Pyrazole starting materials of the Formula XIV may be obtained conventionally, for example by way of Process Variants (a) or (b) as described hereinbefore and/or using procedures that are analogous to those disclosed within the Examples that are set out hereinafter.

(i) For the production of those compounds of the Formula I wherein R³ is a (2-6C)alkanoyl group, the acylation, conveniently in the presence of a suitable base as defined hereinafter, of a 2-aminopyrazole of the Formula XV

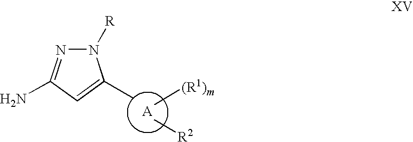

wherein R, Ring A, m, R¹ and R² have any of the meanings defined hereinbefore except that any functional group is protected if necessary, with a (2-6C)alkanoic acid, or a reactive derivative thereof as defined hereinbefore, whereafter any protecting group that is present is removed.

The reaction is conveniently carried out in the presence of a suitable inert solvent or diluent as defined hereinbefore. The reaction is conveniently carried out at a temperature in the range, for example, 0 to 120° C., conveniently at or near 50° C., more conveniently at or near ambient temperature.

Pyrazole starting materials of the Formula XV may be obtained conventionally, for example by way of Process Variants (a) or (b) as described hereinbefore and/or using procedures that are analogous to those disclosed within the Examples that are set out hereinafter.

(j) For the production of those compounds of the Formula I wherein R³ is a N-(1-6C)alkylcarbamoyl group, the coupling, conveniently in the presence of a suitable base as defined hereinafter, of phosgene, or a chemical equivalent thereof, with a 2-aminopyrazole of the Formula XV

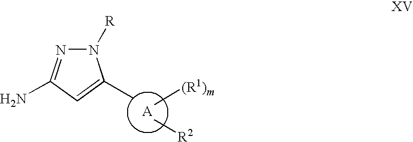

wherein R, Ring A, m, R¹ and R² have any of the meanings defined hereinbefore except that any functional group is protected if necessary, and with a (1-6C)alkylamine, whereafter any protecting group that is present is removed.

A suitable chemical equivalent of phosgene is, for example, a compound of formula

L′-CO-L″

wherein L′ and L″ are suitable leaving groups as defined hereinbefore. For example, a suitable leaving group L′ or L″ is, for example, a halogeno, alkoxy, aryloxy or sulphonyloxy group, for example a chloro, methoxy, phenoxy, methanesulfonyloxy or toluene-4-sulfonyloxy group. For example, a suitable chemical equivalent of phosgene is a formic acid derivative such as phenyl chloroformate. Alternatively, a suitable chemical equivalent of phosgene is a carbonate derivative such as disuccinimido carbonate.

For example, the process may be carried out by reacting a 2-aminopyrazole of the Formula XV with, for example, phenyl chloroformate using known procedures for the preparation of carbamates. The reaction step is conveniently carried out in the presence of a suitable inert solvent or diluent as defined hereinbefore and at a temperature in the range, for example, 0 to 120° C., preferably at or near ambient temperature. The resulting carbamate can be reacted with a (1-6C)alkylamine using known procedures for the preparation of ureido derivatives. This reaction step is conveniently carried out in the presence of a suitable inert solvent or diluent as defined hereinbefore and at a temperature in the range, for example, 0 to 120° C., preferably at or near ambient temperature.

(k) For the production of those compounds of the Formula I wherein R³ is a group of the formula:—

Q⁵-X⁶—

wherein X⁶ is a N(R¹⁰)CO group and Q⁵ has any of the meanings defined hereinbefore, the coupling, conveniently in the presence of a suitable base as defined hereinafter, of phosgene, or a chemical equivalent thereof as defined hereinbefore, with a 2-aminopyrazole of the Formula XV

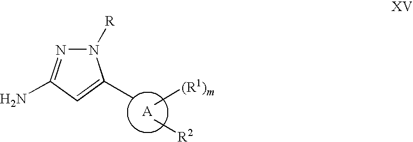

wherein R, Ring A, m, R¹ and R² have any of the meanings defined hereinbefore except that any functional group is protected if necessary, and with an amine of the formula Q⁵NHR¹⁰, wherein Q⁵ and R¹⁰ have any of the meanings defined hereinbefore except that any functional is group is protected if necessary, whereafter any protecting group that is present is removed.

For example, the process may be carried out by reacting a 2-aminopyrazole of the Formula XV with, for example, phenyl chloroformate using known procedures for the preparation of carbamates. The reaction step is conveniently carried out in the presence of a suitable inert solvent or diluent as defined hereinbefore and at a temperature in the range, for example, 0 to 120° C., preferably at or near ambient temperature. The resulting carbamate can be reacted with an amine of the formula Q⁵NHR¹⁰ using known procedures for the preparation of ureido derivatives. This reaction step is conveniently carried out in the presence of a suitable inert solvent or diluent as defined hereinbefore and at a temperature in the range, for example, 0 to 120° C., preferably at or near ambient temperature.

(1) For the production of those compounds of the Formula I wherein R² is a group of the formula:—

—X²-Q²

wherein X² is a N(R⁵) group and Q² is a aryl-(1-6C)alkyl, aryloxy-(1-6C)alkyl, (3-8C)cycloalkyl-(1-6C)alkyl, heteroaryl-(1-6C)alkyl or heterocyclyl-(1-6C)alkyl group, the alkylation, conveniently in the presence of a suitable base as defined hereinbefore, of a compound of the Formula XII

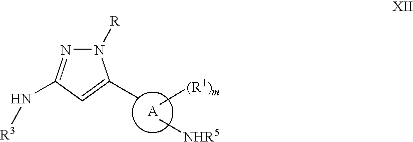

wherein R, Ring A, m, R¹, R³ and R⁵ have any of the meanings defined hereinbefore except that any functional group is protected if necessary, with a compound of the formula:—

L-Q²

wherein L has any of the meanings defined hereinbefore and Q² is a aryl-(1-6C)alkyl, aryloxy-(1-6C)alkyl, (3-8C)cycloalkyl-(1-6C)alkyl, heteroaryl-(1-6C)alkyl or heterocyclyl-(1-6C)alkyl group except that any functional group is protected if necessary, whereafter any protecting group that is present is removed.

The reaction is conveniently carried out in the presence of a suitable inert solvent or diluent as defined hereinbefore. The reaction is conveniently carried out at a temperature in the range, for example, 0 to 150° C., preferably at or near 50° C.

(m) For the production of those compounds of the Formula I wherein R² is a group of the formula:—

—X²-Q²

wherein X² is a N(R⁵) group and Q² is a aryl-methyl, (3-8C)cycloalkyl-methyl, heteroaryl-methyl or heterocyclyl-methyl group, the reaction, conveniently in the presence of a suitable reducing agent, of a compound of the Formula XII

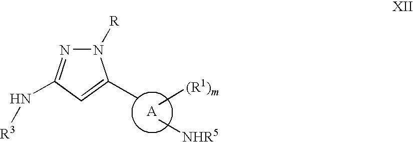

wherein R, Ring A, m, R¹, R³ and R⁵ have any of the meanings defined hereinbefore except that any functional group is protected if necessary, with an aldehyde of the formula:—

OHC-Q²

wherein Q² is a aryl, (3-8C)cycloalkyl, heteroaryl or heterocyclyl group except that any functional group is protected if necessary, whereafter any protecting group that is present is removed.

The reaction is conveniently carried out using known procedures for the reductive amination of aldehydes, for example using a reducing agent such as sodium cyanoborohydride or polymer-bound sodium cyanoborohydride in the presence of a carboxylic acid such as acetic acid. The reaction is conveniently carried out in the presence of a suitable inert solvent or diluent as defined hereinbefore and at a temperature in the range, for example, 0 to 100° C., conveniently at about ambient temperature.

Other suitable reducing agents for the reductive amination reaction include, for example, a hydride reducting agent, for example an alkali metal aluminium hydride such as lithium aluminium hydride or, preferably, an alkali metal borohydride such as sodium borohydride, sodium triethylborohydride, sodium trimethoxyborohydride and sodium triacetoxyborohydride. The reaction is conveniently performed in a suitable inert solvent or diluent, for example tetrahydrofuran and diethyl ether for the more powerful reducing agents such as lithium aluminium hydride, and, for example, methylene chloride or a protic solvent such as methanol and ethanol for the less powerful reducing agents such as sodium triacetoxyborohydride and sodium cyanoborohydride.

The pyrazole derivative of the Formula I may be obtained from the process variants described hereinbefore in the form of the free base or alternatively it may be obtained in the form of a salt with the acid of the formula H-L wherein L has the meaning defined hereinbefore. When it is desired to obtain the free base from the salt, the salt may be treated with a suitable base, for example, an organic amine base such as, for example, pyridine, 2,6-lutidine, collidine, 4-dimethylaminopyridine, triethylamine, morpholine, N-methylmorpholine or diazabicyclo[5.4.0]undec-7-ene, or, for example, an alkali or alkaline earth metal carbonate or hydroxide, for example sodium carbonate, potassium carbonate, calcium carbonate, sodium hydroxide or potassium hydroxide.

When a pharmaceutically-acceptable salt of a pyrazole derivative of the Formula I is required, for example an acid-addition salt, it may be obtained by, for example, reaction of said pyrazole derivative with a suitable acid using a conventional procedure.

When a pharmaceutically-acceptable pro-drug of a pyrazole derivative of the Formula I is required, it may be obtained using a conventional procedure. For example, an in vivo cleavable ester of a pyrazole derivative of the Formula I may be obtained by, for example, reaction of a compound of the Formula I containing a carboxy group with a pharmaceutically-acceptable alcohol or by reaction of a compound of the Formula I containing a hydroxy group with a pharmaceutically-acceptable carboxylic acid. For example, an in vivo cleavable amide of a pyrazole derivative of the Formula I may be obtained by, for example, reaction of a compound of the Formula I containing a carboxy group with a pharmaceutically-acceptable amine or by reaction of a compound of the Formula I containing an amino group with a pharmaceutically-acceptable carboxylic acid.

Many of the intermediates defined herein are novel and these are provided as a further feature of the invention. For example, many compounds of the Formulae XI, XII, XIII, XIV and XV are novel compounds.

### Biological Assays

The following assays can be used to measure the effects of the compounds of the present invention as P13 kinase inhibitors, as mTOR PI kinase-related kinase inhibitors, as inhibitors in vitro of the activation of P13 kinase signalling pathways, as inhibitors in vitro of the proliferation of MDA-MB-468 human breast adenocarcinoma cells, and as inhibitors in vivo of the growth in nude mice of xenografts of MDA-MB-468 carcinoma tissue.

### (a) In Vitro PI3K Enzyme Assay

The assay used AlphaScreen technology (Gray et al., Analytical Biochemistry, 2003, 313: 234-245) to determine the ability of test compounds to inhibit phosphorylation by recombinant Type I PI3K enzymes of the lipid PI(4,5)P2.

DNA fragments encoding human PI3K catalytic and regulatory subunits were isolated from cDNA libraries using standard molecular biology and PCR cloning techniques. The selected DNA fragments were used to generate baculovirus expression vectors. In particular, full length DNA of each of the p110α, p110β and p110δ Type Ia human PI3K p110 isoforms (EMBL Accession Nos. HSU79143, S67334, Y10055 for p110α, p110β and p110δ respectively) were sub-cloned into a pDEST10 vector (Invitrogen Limited, Fountain Drive, Paisley, UK). The vector is a Gateway-adapted version of Fastbac1 containing a 6-His epitope tag. A truncated form of Type Ib human PI3K p110γ isoform corresponding to amino acid residues 144-1102 (EMBL Accession No. X8336A) and the full length human p85α regulatory subunit (EMBL Accession No. HSP13KIN) were also sub-cloned into pFastBac1 vector containing a 6-His epitope tag. The Type Ia p110 constructs were co-expressed with the p85α regulatory subunit. Following expression in the baculovirus system using standard baculovirus expression techniques, expressed proteins were purified using the His epitope tag using standard purification techniques.

DNA corresponding to amino acids 263 to 380 of human general receptor for phosphoinositides (Grp1) PH domain was isolated from a cDNA library using standard molecular biology and PCR cloning techniques. The resultant DNA fragment was sub-cloned into a pGEX 4T1 E. coli expression vector containing a GST epitope tag (Amersham Pharmacia Biotech, Rainham, Essex, UK) as described by Gray et al., Analytical Biochemistry, 2003, 313: 234-245). The GST-tagged Grp1 PH domain was expressed and purified using standard techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one, Brunel Way, Stonehouse, Gloucestershire, UK Catalogue No. 784075). A mixture of each selected recombinant purified PI3K enzyme (15 ng), DiC8-PI(4,5)P2 substrate (40 μM; Cell Signals Inc., Kinnear Road, Columbus, USA, Catalogue No. 901), adenosine triphosphate (ATP; 4 μM) and a buffer solution [comprising Tris-HCl pH7.6 buffer (40 mM, 10 μl), 3-[(3-cholamidopropyl)dimethylammonio]-1-propanesulphonate (CHAPS; 0.04%), dithiothreitol (DTT; 2 mM) and magnesium chloride (10 mM)] was agitated at room temperature for 20 minutes.

Control wells that produced a minimum signal corresponding to maximum enzyme activity were created by using 5% DMSO instead of test compound. Control wells that produced a maximum signal corresponding to fully inhibited enzyme were created by adding wortmannin (6 μM; Calbiochem/Merck Bioscience, Padge Road, Beeston, Nottingham, UK, Catalogue No. 681675) instead of test compound. These assay solutions were also agitated for 20 minutes at room temperature.

Each reaction was stopped by the addition of 10 μl of a mixture of EDTA (100 mM), bovine serum albumin (BSA, 0.045%) and Tris-HCl pH7.6 buffer (40 mM).

Biotinylated-DiC8-PI(3,4,5)P3 (50 mM; Cell Signals Inc., Catalogue No. 107), recombinant purified GST-Grp1 PH protein (2.5 nM) and AlphaScreen Anti-GST donor and acceptor beads (100 ng; Packard Bioscience Limited, Station Road, Pangbourne, Berkshire, UK, Catalogue No. 6760603M) were added and the assay plates were left for about 5 to 20 hours at room temperature in the dark. The resultant signals arising from laser light excitation at 680 nm were read using a Packard AlphaQuest instrument.

PI(3,4,5)P3 is formed in situ as a result of PI3K mediated phosphorylation of P1(4,5)P2. The GST-Grp1 PH domain protein that is associated with AlphaScreen Anti-GST donor beads forms a complex with the biotinylated PI(3,4,5)P3 that is associated with Alphascreen Streptavidn acceptor beads. The enymatically-produced PI(3,4,5)P3 competes with biotinylated PI(3,4,5)P3 for binding to the PH domain protein. Upon laser light excitation at 680 nm, the donor bead: acceptor bead complex produces a signal that can be measured. Accordingly, PI3K enzyme activity to form PI(3,4,5)P3 and subsequent competition with biotinylated PI(3,4,5)P3 results in a reduced signal. In the presence of a PI3K enzyme inhibitor, signal strength is recovered.

PI3K enzyme inhibition for a given test compound was expressed as an IC₅₀ value.

Thereby, the inhibitory properties of compounds of formula (I) against PI3K enzymes, such as the Class Ia PI3K enzymes (e.g. PI3Kalpha, PI3 Kbeta and PI3 Kdelta) and the Class Ib PI3K enzyme (PI3 Kgamma) may be demonstrated.

(b) In Vitro mTOR PI Kinase-Related Kinase Assay

The assay used AlphaScreen technology (Gray et al., Analytical Biochemistry, 2003, 313: 234-245) to determine the ability of test compounds to inhibit phosphorylation by recombinant mTOR.

A C-terminal truncation of mTOR encompassing amino acid residues 1362 to 2549 of mTOR (EMBL Accession No. L34075) was stably expressed as a FLAG-tagged fusion in HEK293 cells as described by Vilella-Bach et al., Journal of Biochemistry, 1999, 274, 4266-4272. The HEK293 FLAG-tagged mTOR (1362-2549) stable cell line was routinely maintained at 37° C. with 5% CO₂ up to a confluency of 70-90% in Dulbecco's modified Eagle's growth medium (DMEM; Invitrogen Limited, Paisley, UK Catalogue No. 41966-029) containing 10% heat-inactivated foetal calf serum (FCS; Sigma, Poole, Dorset, UK, Catalogue No. F0392), 1% L-glutamine (Gibco, Catalogue No. 25030-024) and 2 mg/ml Geneticin (G418 sulphate; Invitrogen Limited, UK Catalogue No. 10131-027). Following expression in the mammalian HEK293 cell line, expressed protein was purified using the FLAG epitope tag using standard purification techniques.

Test compounds were prepared as 10 mM stock solutions in DMSO and diluted into water as required to give a range of final assay concentrations. Aliquots (2 μl) of each compound dilution were placed into a well of a Greiner 384-well low volume (LV) white polystyrene plate (Greiner Bio-one). A 30 μl mixture of recombinant purified mTOR enzyme, 1 μM biotinylated peptide substrate (Biotin-Ahx-Lys-Lys-Ala-Asn-Gln-Val-Phe-Leu-Gly-Phe-Thr-Tyr-Val-Ala-Pro-Ser-Val-Leu-Glu-Ser-Val-Lys-Glu-NH₂; Bachem UK Ltd), ATP (20 μM) and a buffer solution [comprising Tris-HCl pH7.4 buffer (50 mM), EGTA (0.1 mM), bovine serum albumin (0.5 mg/ml), DTT (1.25 mM) and manganese chloride (10 mM)] was agitated at room temperature for 90 minutes.

Control wells that produced a maximum signal corresponding to maximum enzyme activity were created by using 5% DMSO instead of test compound. Control wells that produced a minimum signal corresponding to fully inhibited enzyme were created by adding EDTA (83 mM) instead of test compound. These assay solutions were incubated for 2 hours at room temperature.

Each reaction was stopped by the addition of 10 μl of a mixture of EDTA (50 mM), bovine serum albumin (BSA; 0.5 mg/ml) and Tris-HCl pH7.4 buffer (50 mM) containing p70 S6 Kinase (T389) 1A5 Monoclonal Antibody (Cell Signalling Technology, Catalogue No. 9206B) and AlphaScreen Streptavidin donor and Protein A acceptor beads (200 ng; Perkin Elmer, Catalogue No. 6760002B and 6760137R respectively) were added and the assay plates were left for about 20 hours at room temperature in the dark. The resultant signals arising from laser light excitation at 680 nm were read using a Packard Envision instrument.

Phosphorylated biotinylated peptide is formed in situ as a result of mTOR mediated phosphorylation. The phosphorylated biotinylated peptide that is associated with AlphaScreen Streptavidin donor beads forms a complex with the p70 S6 Kinase (T389) 1A5 Monoclonal Antibody that is associated with Alphascreen Protein A acceptor beads. Upon laser light excitation at 680 nm, the donor bead: acceptor bead complex produces a signal that can be measured. Accordingly, the presence of mTOR kinase activity results in an assay signal. In the presence of an mTOR kinase inhibitor, signal strength is reduced.

mTOR enzyme inhibition for a given test compound was expressed as an IC₅₀ value.

### (c) In Vitro Phospho-Ser473 Akt Assay

This assay determines the ability of test compounds to inhibit phosphorylation of Serine 473 in Akt as assessed using Acumen Explorer technology (TTP LabTech Limited, Royston, Herts, SG8 6EE, UK), a plate reader that can be used to rapidly quantitate features of images generated by laser-scanning.

A MDA-MB-468 human breast adenocarcinoma cell line (LGC Promochem, Teddington, Middlesex, UK, Catalogue No. HTB-132) was routinely maintained at 37° C. with 5% CO₂ up to a confluency of 70-90% in DMEM containing 10% FCS and 1% L-glutamine.

For the assay, the cells were detached from the culture flask using ‘Accutase’ (Innovative Cell Technologies Inc., San Diego, Calif., USA; Catalogue No. AT104) using standard tissue culture methods and resuspended in media to give 5.5×10⁴ cells per ml. Aliquots (90 μl) were seeded into each of the inner 60 wells of a black ‘Costar’ 96-well plate (Corning Inc., NY, USA; Catalogue No. 3904) to give a density of 5000 cells per well. Aliquots (90 μl) of culture media were placed in the outer wells to prevent edge effects. [An alternative cell handling procedure involved the maintenance of the cells in a ‘SelecT’ robotic device (The Automation Partnership, Royston, Herts SG8 5WY, UK). Cells were resuspended in media to give 5×10⁴ cells per ml. Aliquots (100 μl) were seeded into the wells of a black ‘Costar’ 96-well plate.] The cells were incubated overnight at 37° C. with 5% CO₂ to allow them to adhere.

On day 2, the cells were treated with test compounds. Test compounds were prepared as 10 mM stock solutions in DMSO and serially diluted as required with DMSO and with growth media to give a range of concentrations that were 10-fold the required final test concentrations. Aliquots (10 μl) of each compound dilution were placed in duplicate wells to give the final required concentrations. As a minimum response control, each plate contained wells having a final concentration of 30 μM LY294002 (Calbiochem, Beeston, UK, Catalogue No. 440202). As a maximum response control, wells contained 0.5% DMSO instead of test compound. [An alternative cell treatment procedure involved the transfer of test compounds to the wells using an ‘Echo 550’ liquid dispenser (Labcyte Inc., Sunnyvale, Calif. 94089, USA). Test compounds were prepared as 10 mM stock solutions in DMSO and aliquots (40 μl) of each compound were dispensed into one well of a quadrant of wells within a 384-well plate (Labcyte Inc., Catalogue No. P-05525-CV1). Four concentrations of each compound were prepared in each quadrant of wells in the 384-well plate using a ‘Hydra II’ pipettor (Matrix Technologies Corporation, Handforth SK9 3LP, UK). Using a ‘Quadra Tower’ liquid pipetting system (Tomtec Inc., Hamden, Conn. 06514, USA) and the ‘Echo 550’ liquid dispenser, the required concentration of each compound was placed in specific wells in duplicate.] The treated cells were incubated for 2 hours at 37° C. with 5% CO₂.

Following incubation, the contents of the plates were fixed by treatment with a 1.6% aqueous formaldehyde solution (Sigma, Poole, Dorset, UK, Catalogue No. F1635) at room temperature for 30 minutes.

All subsequent aspiration and washing steps were carried out using a Tecan 96-well plate washer (aspiration speed 10 mm/sec). The fixing solution was removed and the contents of the plates were washed with phosphate-buffered saline (PBS; 50 μl; such as that available from Gibco, Catalogue No. 10010015). The contents of the plates were treated at room temperature for 1 hour with an aliquot (50 μl) of a cell permeabilisation/blocking buffer consisting of a mixture of PBS, 0.5% Tween-20 and 5% dried skimmed milk [‘Marvel’ (registered trade mark); Premier Beverages, Stafford, GB]. The permeabilisation/blocking buffer caused the cell wall to be partially degraded to allow immunostaining to proceed whilst blocking non-specific binding sites. The buffer was removed and the cells were incubated for 16 hours at 4° C. with rabbit anti-phospho-Akt (Ser473) antibody solution (50 μl per well; Cell Signaling Technology Inc., Hitchin, Herts, U.K., Catalogue No. 3787) that had been diluted 1:500 in ‘blocking’ buffer consisting of a mixture of PBS, 0.5% Tween-20 and 5% dried skimmed milk. Cells were washed three times in a mixture of PBS and 0.05% Tween-20. Subsequently, cells were incubated for 1 hour at 4° C. with Alexafluor488 labelled goat anti-rabbit IgG (501 per well; Molecular Probes, Invitrogen Limited, Paisley, UK, Catalogue No. A11008) that had been diluted 1:500 in ‘blocking’ buffer. Cells were washed 3 times with a mixture of PBS and 0.05% Tween-20. An aliquot of PBS containing 1.6% aqueous formaldehyde (50 μl) was added to each well. After 15 minutes, the formaldehyde was removed and each of the wells was washed with PBS (100 μl). An aliquot of PBS (50 μl) was added to each well and the plates were sealed with black plate sealers and the fluorescence signal was detected and analysed.

Fluorescence dose response data obtained with each compound were analysed and the degree of inhibition of Serine 473 in Akt was expressed as an IC₅₀ value.

### (d) In Vitro MDA-MB-468 Human Breast Adenocarcinoma Proliferation Assay

This assay determines the ability of test compounds to inhibit cell proliferation, as assessed by the extent of metabolism by living cells of a tetrazolium dye. A MDA-MB-468 human breast carcinoma cell line (ATCC, Catalogue No. HTB-132) was routinely maintained as described in Biological Assay (c) hereinbefore except that the growth medium did not contain phenol red.

For the proliferation assay, the cells were detached from the culture flask using ‘Accutase’ and, at a density of 4000 cells per well in 100 μl of complete growth medium, the cells were placed in wells in a ‘Costar’ 96-well tissue culture-treated plate (Corning Inc., Catalogue No. 3598). Aliquots (100 μl) per well of growth medium were added to some wells to provide blank values for the colorometric measurement. The cells were incubated overnight at 37° C. with 5% CO₂ to allow them to adhere.

Sufficient phenazine ethosulphate (PES, Sigma Catalogue No. P4544) was added to a 1.9 mg/ml solution of 3-(4,5-dimethylthiazol-2-yl)-5-(3 carboxymethoxyphenyl)-2-(4-sulphophenyl)-2H-tetrazolium salt (MTS; Promega UK, Southampton SO16 7NS, UK; Catalogue No. G1111) to give a 0.3 mM PES solution. An aliquot (20 μl) of the resultant MTS/PES solution was added to each well of one plate. The cells were incubated for 2 hours at 37° C. with 5% CO₂ and the optical density was measured on a plate reader using a wavelength of 492 nm. The relative cell number at the commencement of the assay was thereby measured.

Test compounds were prepared as 10 mM stock solutions in DMSO and serially diluted with growth medium to give a range of test concentrations. An aliquot (50 μl) of each compound dilution was placed in a well in the 96-well plates. Each plate contained control wells without test compound. With the exception of wells containing the plate blanks, the outer wells on each 96-well plate were not used. The cells were incubated for 72 hours at 37° C. with 5% CO₂. An aliquot (30 μl) of the MTS/PES solution was added to each well and the cells were incubated for 2 hours at 37° C. with 5% CO₂. The optical density was measured on a plate reader using a wavelength of 492 nm.

Dose response data were obtained for each test compound and the degree of inhibition of MDA-MB-468 cell growth was expressed as an IC₅₀ value.

### (e) In Vivo MDA-MB-468 Xenograft Growth Assay

This test measures the ability of compounds to inhibit the growth of MDA-MB-468 human breast adenocarcinoma cells grow as a tumour in athymic nude mice (Alderley Park nu/nu strain). A total of about 5×10⁶ MDA-MB-468 cells in matrigel (Beckton Dickinson Catalogue No. 40234) are injected subcutaneously into the left flank of each test mouse and the resultant tumours are allowed to grow for about 14 days. Tumour size is measured twice weekly using callipers and a theoretical volume is calculated. Animals are selected to provide control and treatment groups of approximately equal average tumour volume. Test compounds are prepared as a ball-milled suspension in 1% polysorbate vehicle and dosed orally once daily for a period of about 28 days. The effect on tumour growth is assessed.

Although the pharmacological properties of the compounds of the Formula I vary with structural change as expected, in general activity possessed by many of the compounds of the Formula I, may be demonstrated at the following concentrations or doses in one or more of the above tests (a), (b), (c), (d) and (e):—
Test (a):—IC₅₀ versus p110α Type Ia human PI3K in the range, for example, 0.1-20 μM;
Test (b):—IC₅₀ versus mTOR PI kinase-related kinase in the range, for example, 0.1-40 μM;
Test (c):—IC₅₀ in the range, for example, 0.1-50 μM;
Test (d):—IC₅₀ in the range, for example, 0.1-50 μM;
Test (e):—activity in the range, for example, 1-200 mg/kg/day.

For example, the pyrazole compound disclosed within Example 1 possesses activity in Test (a) with an IC₅₀ versus p110α Type Ia human PI3K of approximately 5 μM, and in Test (b) with an IC₅₀ versus mTOR PI kinase-related kinase of approximately 40 μM.

For example, the pyrazole compound disclosed within Example 3 possesses activity in Test (a) with an IC₅₀ versus p110α Type Ia human PI3K of approximately 0.5 μM, and in Test (b) with an IC₅₀ versus mTOR PI kinase-related kinase of approximately 2 μM.

No untoward toxicological effects are expected when a compound of Formula I, or a pharmaceutically-acceptable salt thereof, as defined hereinbefore is administered at the dosage ranges defined hereinafter.

According to a further aspect of the invention there is provided a pharmaceutical composition which comprises a pyrazole derivative of the Formula I, or a pharmaceutically-acceptable salt thereof, as defined hereinbefore in association with a pharmaceutically-acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intraperitoneal or intramuscular dosing) or for rectal administration (for example as a suppository).

The compositions of the invention may be obtained by conventional procedures using conventional pharmaceutical excipients, well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the host treated and the particular route of administration. For example, a formulation intended for oral administration to humans will generally contain, for example, from 1 mg to 1 g of active agent (more suitably from 1 to 250 mg, for example from 1 to 100 mg) compounded with an appropriate and convenient amount of excipients which may vary from about 5 to about 98 percent by weight of the total composition.

The size of the dose for therapeutic or prophylactic purposes of a compound of the Formula I will naturally vary according to the nature and severity of the disease state, the age and sex of the animal or patient and the route of administration, according to well known principles of medicine.

In using a compound of the Formula I for therapeutic or prophylactic purposes it will generally be administered so that a daily dose in the range, for example, 1 mg/kg to 100 mg/kg body weight is received, given if required in divided doses. In general, lower doses will be administered when a parenteral route is employed. Thus, for example, for intravenous administration, a dose in the range, for example, 1 mg/kg to 25 mg/kg body weight will generally be used. Similarly, for administration by inhalation, a dose in the range, for example, 1 mg/kg to 25 mg/kg body weight will be used. Oral administration is however preferred, particularly in tablet form. Typically, unit dosage forms will contain about 10 mg to 0.5 g of a compound of this invention.

As stated above, it is known that PI3K enzymes contribute to tumourigenesis by one or more of the effects of mediating proliferation of cancer and other cells, mediating angiogenic events and mediating the motility, migration and invasiveness of cancer cells. We have found that the pyrazole derivatives of the present invention possess potent anti-tumour activity which it is believed is obtained by way of inhibition of one or more of the Class I PI3K enzymes (such as the Class Ia PI3K enzymes and/or the Class Ib PI3K enzyme) and/or a mTOR kinase (such as a mTOR PI kinase-related kinase) that are involved in the signal transduction steps which lead to the proliferation and survival of tumour cells and the invasiveness and migratory ability of metastasising tumour cells.

Accordingly, the derivatives of the present invention are of value as anti-tumour agents, in particular as selective inhibitors of the proliferation, survival, motility, dissemination and invasiveness of mammalian cancer cells leading to inhibition of tumour growth and survival and to inhibition of metastatic tumour growth. Particularly, the pyrazole derivatives of the present invention are of value as anti-proliferative and anti-invasive agents in the containment and/or treatment of solid tumour disease. Particularly, the compounds of the present invention are expected to be useful in the prevention or treatment of those tumours which are sensitive to inhibition of one or more of the multiple PI3K enzymes such as the Class Ia PI3K enzymes and the Class Ib PI3K enzyme that are involved in the signal transduction steps which lead to the proliferation and survival of tumour cells and the migratory ability and invasiveness of metastasising tumour cells. Further, the compounds of the present invention are expected to be useful in the prevention or treatment of those tumours which are mediated alone or in part by inhibition of PI3K enzymes such as the Class Ia PI3K enzymes and the Class Ib PI3K enzyme, i.e. the compounds may be used to produce a PI3K enzyme inhibitory effect in a warm-blooded animal in need of such treatment.

As stated hereinbefore, inhibitors of PI3K enzymes should be of therapeutic value for treatment of, for example, cancer of the breast, colorectum, lung (including small cell lung cancer, non-small cell lung cancer and bronchioalveolar cancer) and prostate, and of cancer of the bile duct, bone, bladder, head and neck, kidney, liver, gastrointestinal tissue, oesophagus, ovary, pancreas, skin, testes, thyroid, uterus, cervix and vulva, and of leukaemias [including acute lymphoctic leukaemia (ALL) and chronic myelogenous leukaemia (CML)], multiple myeloma and lymphomas.

According to a further aspect of the invention there is provided a pyrazole derivative of the Formula I, or a pharmaceutically-acceptable salt thereof, as defined hereinbefore for use as a medicament in a warm-blooded animal such as man.

According to a further aspect of the invention, there is provided a pyrazole derivative of the Formula I, or a pharmaceutically-acceptable salt thereof, as defined hereinbefore for use in the production of an anti-proliferative effect in a warm-blooded animal such as man.

According to a further feature of this aspect of the invention there is provided a pyrazole derivative of the Formula I, or a pharmaceutically-acceptable salt thereof, as defined hereinbefore for use in a warm-blooded animal such as man as an anti-invasive agent in the containment and/or treatment of solid tumour disease.

According to a further aspect of the invention, there is provided the use of a pyrazole derivative of the Formula I, or a pharmaceutically-acceptable salt thereof, as defined hereinbefore for the production of an anti-proliferative effect in a warm-blooded animal such as man.

According to a further feature of this aspect of the invention there is provided the use of a pyrazole derivative of the Formula I, or a pharmaceutically-acceptable salt thereof, as defined hereinbefore in the manufacture of a medicament for use in the production of an anti-proliferative effect in a warm-blooded animal such as man.

According to a further feature of this aspect of the invention there is provided the use of a pyrazole derivative of the Formula I, or a pharmaceutically-acceptable salt thereof, as defined hereinbefore in the manufacture of a medicament for use in a warm-blooded animal such as man as an anti-invasive agent in the containment and/or treatment of solid tumour disease.

According to a further feature of this aspect of the invention there is provided a method for producing an anti-proliferative effect in a warm-blooded animal, such as man, in need of such treatment which comprises administering to said animal an effective amount of a pyrazole derivative of the Formula I, or a pharmaceutically-acceptable salt thereof, as defined hereinbefore.

According to a further feature of this aspect of the invention there is provided a method for producing an anti-invasive effect by the containment and/or treatment of solid tumour disease in a warm-blooded animal, such as man, in need of such treatment which comprises administering to said animal an effective amount of a pyrazole derivative of the Formula I, or a pharmaceutically-acceptable salt thereof, as defined hereinbefore.

According to a further aspect of the invention there is provided the use of a pyrazole derivative of the Formula I, or a pharmaceutically-acceptable salt thereof, as defined hereinbefore in the manufacture of a medicament for use in the prevention or treatment of solid tumour disease in a warm-blooded animal such as man.

According to a further feature of this aspect of the invention there is provided a method for the prevention or treatment of solid tumour disease in a warm-blooded animal, such as man, in need of such treatment which comprises administering to said animal an effective amount of a pyrazole derivative of the Formula I, or a pharmaceutically-acceptable salt thereof, as defined hereinbefore.

According to a further aspect of the invention there is provided a pyrazole derivative of the Formula I, or a pharmaceutically-acceptable salt thereof, as defined hereinbefore for use in the prevention or treatment of those tumours which are sensitive to inhibition of PI3K enzymes (such as the Class Ia enzymes and/or the Class Ib PI3K enzyme) and/or a mTOR kinase (such as a mTOR PI kinase-related kinase) that are involved in the signal transduction steps which lead to the proliferation, survival, invasiveness and migratory ability of tumour cells.

According to a further feature of this aspect of the invention there is provided the use of a pyrazole derivative of the Formula I, or a pharmaceutically-acceptable salt thereof, as defined hereinbefore in the manufacture of a medicament for use in the prevention or treatment of those tumours which are sensitive to inhibition of PI3K enzymes (such as the Class Ia enzymes and/or the Class Ib PI3K enzyme) and/or a mTOR kinase (such as a mTOR PI kinase-related kinase) that are involved in the signal transduction steps which lead to the proliferation, survival, invasiveness and migratory ability of tumour cells.

According to a further feature of this aspect of the invention there is provided a method for the prevention or treatment of those tumours which are sensitive to inhibition of PI3K enzymes (such as the Class Ia enzymes and/or the Class Ib PI3K enzyme) and/or a mTOR kinase (such as a mTOR PI kinase-related kinase) that are involved in the signal transduction steps which lead to the proliferation, survival, invasiveness and migratory ability of tumour cells which comprises administering to said animal an effective amount of a pyrazole derivative of the Formula I, or a pharmaceutically-acceptable salt thereof, as defined hereinbefore.

According to a further aspect of the invention there is provided a pyrazole derivative of the Formula I, or a pharmaceutically-acceptable salt thereof, as defined hereinbefore for use in providing a PI3K enzyme inhibitory effect (such as a Class Ia PI3K enzyme or Class Ib PI3K enzyme inhibitory effect) and/or a mTOR kinase inhibitory effect (such as a mTOR PI kinase-related kinase inhibitory effect).

According to a further feature of this aspect of the invention there is provided the use of a pyrazole derivative of the Formula I, or a pharmaceutically-acceptable salt thereof, as defined hereinbefore in the manufacture of a medicament for use in providing a PI3K enzyme inhibitory effect (such as a Class Ia PI3K enzyme or Class Ib PI3K enzyme inhibitory effect) and/or a mTOR kinase inhibitory effect (such as a mTOR PI kinase-related kinase inhibitory effect).

According to a further aspect of the invention there is also provided a method for providing a PI3K enzyme inhibitory effect (such as a Class Ia PI3K enzyme or Class Ib PI3K enzyme inhibitory effect) and/or a mTOR kinase inhibitory effect (such as a mTOR PI kinase-related kinase inhibitory effect) which comprises administering an effective amount of a pyrazole derivative of the Formula I, or a pharmaceutically-acceptable salt thereof, as defined hereinbefore.

As stated hereinbefore, certain compounds of the present invention, possess substantially better potency against Class Ia PI3K enzymes and against a mTOR kinase (such as a mTOR PI kinase-related kinase) than against EGF receptor tyrosine kinase, VEGF receptor tyrosine kinase or Src non-receptor tyrosine kinase enzymes. Such compounds possess sufficient potency against Class Ia PI3K enzymes and mTOR kinases that they may be used in an amount sufficient to inhibit Class Ia PI3K enzymes and nTOR kinases whilst demonstrating little activity against EGF receptor tyrosine kinase, VEGF receptor tyrosine kinase or Src non-receptor tyrosine kinase enzymes. Such compounds are likely to be useful for the selective inhibition of Class Ia PI3K enzymes and mTOR kinases and are likely to be useful for the effective treatment of, for example, Class Ia PI3K enzyme driven tumours.

According to this aspect of the invention there is provided a pyrazole derivative of the Formula I, or a pharmaceutically-acceptable salt thereof, as defined hereinbefore for use in providing a selective Class Ia PI3K enzyme and/or mTOR kinase inhibitory effect.

According to a further feature of this aspect of the invention there is provided the use of a pyrazole derivative of the Formula I, or a pharmaceutically-acceptable salt thereof, as defined hereinbefore in the manufacture of a medicament for use in providing a selective Class Ia PI3K enzyme and/or mTOR kinase inhibitory effect.

According to a further aspect of the invention there is also provided a method for providing a selective Class Ia PI3K enzyme and/or mTOR kinase inhibitory effect which comprises administering an effective amount of a pyrazole derivative of the Formula I, or a pharmaceutically-acceptable salt thereof, as defined hereinbefore.

By “a selective Class Ia PI3K enzyme inhibitory effect” is meant that the pyrazole derivatives of the Formula I are more potent against Class Ia PI3K enzymes and/or mTOR kinases than against many other kinase enzymes. In particular, some of the compounds according to the invention are more potent against Class Ia PI3K enzymes and/or mTOR kinases than against other kinases such as other receptor or non-receptor tyrosine kinases or serine/threonine kinases. For example, a selective Class Ia PI3K enzyme inhibitor according to the invention is at least 5 times more potent, preferably at least 10 times more potent, more preferably at least 100 times more potent, against Class Ia PI3K enzymes than against other kinases such as EGF receptor tyrosine kinase, VEGF receptor tyrosine kinases or Src non-receptor tyrosine kinases.

According to a further feature of the invention there is provided a pyrazole derivative of the Formula I, or a pharmaceutically-acceptable salt thereof, as defined hereinbefore for use in the treatment of cancer of the breast, colorectum, lung (including small cell lung cancer, non-small cell lung cancer and bronchioalveolar cancer) and prostate.

According to a further feature of this aspect of the invention there is provided a pyrazole derivative of the Formula I, or a pharmaceutically-acceptable salt thereof, as defined hereinbefore for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of this aspect of the invention there is provided the use of a pyrazole derivative of the Formula I, or a pharmaceutically-acceptable salt thereof, as defined hereinbefore in the manufacture of a medicament for use in the treatment of cancer of the breast, colorectum, lung (including small cell lung cancer, non-small cell lung cancer and bronchioalveolar cancer) and prostate.

According to a further feature of this aspect of the invention there is provided the use of a pyrazole derivative of the Formula I, or a pharmaceutically-acceptable salt thereof, as defined hereinbefore in the manufacture of a medicament for use in the treatment of cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas.

According to a further feature of this aspect of the invention there is provided a method for treating cancer of the breast, colorectum, lung (including small cell lung cancer, non-small cell lung cancer and bronchioalveolar cancer) and prostate in a warm blooded animal such as man that is in need of such treatment which comprises administering an effective amount of a pyrazole derivative of the Formula I, or a pharmaceutically-acceptable salt thereof, as defined hereinbefore.

According to a further feature of this aspect of the invention there is provided a method to for treating cancer of the bile duct, bone, bladder, head and neck, kidney, liver, gastrointestinal tissue, oesophagus, ovary, pancreas, skin, testes, thyroid, uterus, cervix and vulva, and of leukaemias (including ALL and CML), multiple myeloma and lymphomas in a warm blooded animal such as man that is in need of such treatment which comprises administering an effective amount of a pyrazole derivative of the Formula I, or a pharmaceutically-acceptable salt thereof, as defined hereinbefore.

As stated hereinbefore, the in vivo effects of a compound of the Formula I may be exerted in part by one or more metabolites that are formed within the human or animal body after administration of a compound of the Formula I.

The anti-cancer treatment defined hereinbefore may be applied as a sole therapy or may involve, in addition to the pyrazole derivative of the invention, conventional surgery or radiotherapy or chemotherapy. Such chemotherapy may include one or more of the following categories of anti-tumour agents:—

(i) other antiproliferative/antineoplastic drugs and combinations thereof, as used in medical oncology, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example antifolates such as fluoropyrimidines like 5-fluorouracil and tegafur, raltitrexed, methotrexate, cytosine arabinoside, hydroxyurea and gemcitabine); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine, taxoids like taxol and taxotere, and polo kinase inhibitors); and topoisomerase inhibitors (for example epipodophyllotoxins like etoposide and teniposide, amsacrine, topotecan and camptothecin);
(ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341) and bosutinib (SKI-606), and metalloproteinase inhibitors like marimastat and inhibitors of urokinase plasminogen activator receptor function];
(iv) inhibitors of growth factor function: for example such inhibitors include growth factor antibodies and growth factor receptor antibodies [for example the anti-erbB2 antibody trastuzumab and the anti-erbB1 antibodies cetuximab (C225) and panitumumab]; such inhibitors also include, for example, tyrosine kinase inhibitors [for example inhibitors of the epidermal growth factor family (for example EGFR family tyrosine kinase inhibitors such as gefitinib (ZD1839), erlotinib (OSI-774) and CI 1033, and erbB2 tyrosine kinase inhibitors such as lapatinib), inhibitors of the hepatocyte growth factor family, inhibitors of the insulin growth factor receptor, inhibitors of the platelet-derived growth factor family and/or bcr/abl kinase such as imatinib, dasatinib (BMS-354825) and nilotinib (AMN107), inhibitors of cell signalling through MEK, AKT, P13, c-kit, Flt3, CSF-1R and/or aurora kinases]; such inhibitors also include cyclin dependent kinase inhibitors including CDK2 and CDK4 inhibitors; and such inhibitors also include, for example, inhibitors of serine/threonine kinases (for example Ras/Raf signalling inhibitors such as farnesyl transferase inhibitors, for example sorafenib (BAY 43-9006), tipifarnib (R115777) and lonafarnib (SCH66336);
(v) antiangiogenic agents such as those which inhibit the effects of vascular endothelial growth factor, [for example an anti-vascular endothelial cell growth factor antibody such as bevacizumab (Avastin™) or, for example, a VEGF receptor tyrosine kinase inhibitor such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), or, for example, a compound that works by another mechanism (for example linomide, inhibitors of integrin αvβ3 function and angiostatin)];
(vi) vascular damaging agents such as Combretastatin A4 and compounds disclosed in International Patent Applications WO 99/02166, WO 00/40529, WO 00/41669, WO 01/92224, WO 02/04434 and WO 02/08213;
(vii) antisense therapies, for example those which are directed to the targets listed above, such as ISIS 2503, an anti-ras antisense;
(viii) gene therapy approaches, including for example approaches to replace aberrant genes to such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a pharmaceutical product comprising a pyrazole derivative of the Formula I as defined hereinbefore and an additional anti-tumour agent as defined hereinbefore for the conjoint treatment of cancer.

Although the compounds of the Formula I are primarily of value as therapeutic agents for use in warm-blooded animals (including man), they are also useful whenever it is required to inhibit the effects of PI3K enzymes and/or mTOR kinases. Thus, they are useful as pharmacological standards for use in the development of new biological tests and in the search for new pharmacological agents.

The invention will now be illustrated in the following Examples in which, generally:

(i) operations were carried out at ambient temperature, i.e. in the range 17 to 25° C. and under an atmosphere of an inert gas such as nitrogen or argon unless otherwise stated;

(ii) reactions conducted under microwave radiation may be performed using an instrument such as a ‘Smith Synthesiser’ (300 KWatts) on either the normal or high setting, which instrument makes use of a temperature probe to adjust the microwave power output automatically in order to maintain the required temperature; alternatively an ‘Emrys Optimizer’ microwave instrument may be used;

(iii) in general, the course of reactions was followed by thin layer chromatography (TLC) and/or analytical high pressure liquid chromatography (HPLC); the reaction times that are given are not necessarily the minimum attainable;

(iv) when necessary, organic solutions were dried over anhydrous magnesium sulphate, work-up procedures were carried out after removal of residual solids by filtration, evaporations were carried out by rotary evaporation in vacuo;

(v) yields, where present, are not necessarily the maximum attainable, and, when necessary, reactions were repeated if a larger amount of the reaction product was required;

(vi) in general, the structures of the end-products of the Formula I were confirmed by proton nuclear magnetic resonance (¹H NMR) and/or mass spectral techniques; electrospray mass spectral data were obtained using a Waters ZMD or Waters ZQ LC/mass spectrometer acquiring both positive and negative ion data, generally, only ions relating to the parent structure are reported; proton NMR chemical shift values were measured on the delta scale using either a Bruker Spectrospin DPX300 spectrometer operating at a field strength of 300 MHz or a Bruker Avance spectrometer operating at a field strength of 400 MHz; the following abbreviations have been used: s, singlet; d, doublet; t, triplet; q, quartet; m, multiplet; br, broad;

(vii) unless stated otherwise compounds containing an asymmetric carbon and/or sulphur atom were not resolved;

(viii) intermediates were not necessarily fully purified but their structures and purity were assessed by TLC, analytical HPLC, infra-red (IR) and/or NMR analysis;

(ix) unless otherwise stated, column chromatography (by the flash procedure) and medium pressure liquid chromatography (MPLC) were performed on Merck Kieselgel silica (Art. 9385);

(x) preparative HPLC was performed on C18 reversed-phase silica, for example on a Phenomenex ‘Gemini’ C18 column (5 microns silica, 20 mm diameter, 100 mm length) or a Waters ‘Xterra’ C18 column (5 microns silica, 19 mm diameter, 100 mm length) using decreasingly polar solvent mixtures as eluent, for example decreasingly polar mixtures of water (containing 0.05% to 2% aqueous formic acid) and acetonitrile, or, for example, decreasingly polar mixtures of water (containing 0.05% to 2% aqueous ammonium hydroxide) and acetonitrile;

(xi) analytical HPLC methods selected from those presented below were used; in general, reversed-phase silica was used with a flow rate of about 1 ml per minute and detection was by Electrospray Mass Spectrometry and by UV absorbance using a diode array detector over a wavelength of 220 to 300 nm; for each method Solvent A was water (optionally containing a small amount of formic or acetic acid or a small amount of aqueous ammonium hydroxide) and Solvent B was acetonitrile:—

Method A1: Phenomenex ‘Gemini’ C18 column (5 microns silica, 2 mm diameter, 50 mm length) using a Solvent A comprising 0.1% aqueous formic acid and a Solvent B of acetonitrile, a solvent gradient over 4 minutes from a 19:1 mixture of Solvents A and B to a 1:19 mixture of Solvents A and B, and a flow rate of 1.2 ml per minute;

Method B1: Phenomenex ‘Gemini’ C18 column (5 microns silica, 2 mm diameter, 50 mm length) using a Solvent A comprising 0.1% aqueous ammonium hydroxide and a Solvent B of acetonitrile, a solvent gradient over 4 minutes from a 19:1 mixture of Solvents A and B to a 1:19 mixture of Solvents A and B and a flow rate of 1.2 ml per minute;

(xii) where certain compounds are obtained as an acid-addition salt, for example a mono-hydrochloride salt or a di-hydrochloride salt, the stoichiometry of the salt is based on the number and nature of the basic groups in the compound, the exact stoichiometry of the salt is generally not determined, for example by means of elemental analysis data;

(xiii) the following abbreviations have been used:—
DMSO dimethylsulphoxide
THF tetrahydrofuran

### EXAMPLE 1

### N-[5-(5-methanesulphonamidopyridin-3-yl)-1-methyl-1H-pyrazol-3-yl]acetamide

Methanesulphonyl chloride (0.047 ml) was added to a stirred mixture of N-[5-(5-aminopyridin-3-yl)-1-methyl-1H-pyrazol-3-yl]acetamide (0.1 g), triethylamine (0.084 ml) and THF (2 ml) and the resultant mixture was stirred at ambient temperature for 30 minutes. Further portions of methanesulphonyl chloride (0.047 ml) and of triethylamine (0.084 ml) were added and the reaction mixture was stirred at ambient temperature for a further 30 minutes. Pyrrolidine (1 ml) was added and the mixture was stirred at ambient temperature for 30 minutes. The mixture was evaporated and the residue was purified by column chromatography on silica using a 10:1 mixture of methylene chloride and methanol as eluent. The material so obtained was triturated under diethyl ether. The resultant solid was isolated, washed with diethyl ether and dried under vacuum. There was thus obtained the title compound (0.102 g); ¹H NMR Spectrum: (DMSOd₆) 2.02 (s, 3H), 3.14 (s, 3H), 3.79 (s, 3H), 6.72 (s, 1H), 7.72 (t, 1H), 8.49 (d, 1H), 8.51 (d, 1H), 10.17 (s, 1H), 10.46 (s, 1H); Mass Spectrum: M+H⁺ 310.

The N-[5-(5-aminopyridin-3-yl)-1-methyl-1H-pyrazol-3-yl]acetamide used as a starting material was prepared as follows:—

3-Acetyl-5-bromopyridine (0.6 g) was added portionwise to a stirred solution of sodium methoxide (0.324 g) and dimethyl oxalate (0.708 g) in methanol (10 ml) and the resultant mixture was stirred at ambient temperature for 2 hours. The precipitate was isolated, washed in turn with methanol and diethyl ether and dried under vacuum. There was thus obtained methyl 4-(5-bromopyridin-3-yl)-2,4-dioxobutanoate, sodium salt, (0.730 g); Mass Spectrum: M+H⁺ 286.

A mixture of the material so obtained, methyl hydrazine (0.139 ml) and glacial acetic acid (10 ml) was stirred and heated to 110° C. for 2 hours. The bulk of the acetic acid was evaporated and the residue was treated with water (30 ml) and extracted with an 17:3 mixture of methylene chloride and methanol. The organic solution was washed with water, dried over magnesium sulphate and evaporated. The residue was purified by column chromatography on silica using 50-60% ethyl acetate in isohexane as eluent. There was thus obtained methyl 5-(5-bromopyridin-3-yl)-1-methyl-1H-pyrazole-3-carboxylate (0.391 g); ¹H NMR Spectrum: (DMSOd₆) 3.81 (s, 3H), 3.97 (s, 3H), 7.1 (s, 1H), 8.35 (t, 1H), 8.79 (t, 2H).

After repetition of the previous steps, a mixture of methyl 5-(5-bromopyridin-3-yl)-1-methyl-1H-pyrazole-3-carboxylate (0.6 g), 2N aqueous sodium hydroxide (2.5 ml) and methanol (10 ml) was stirred at ambient temperature for 1 hour, followed by heating to 50° C. for 10 minutes. The resultant mixture was cooled to ambient temperature and acidified to pH4 by the addition of dilute aqueous hydrochloric acid. The resultant precipitate was isolated, washed with water and dried under high vacuum. There was thus obtained 5-(5-bromopyridin-3-yl)-1-methyl-1H-pyrazole-3-carboxylic acid (0.425 g); Mass Spectrum: M+H⁺ 282.

After repetition of the previous step, a mixture of 5-(5-bromopyridin-3-yl)-1-methyl-1H-pyrazole-3-carboxylic acid (1.7 g), diphenylphosphoryl azide (1.36 ml), triethylamine (0.918 ml), tert-butanol (3.2 ml) and 1,4-dioxane (10 ml) was stirred and heated to 100° C. for 1 hour. The mixture was diluted with ethyl acetate (40 ml) and washed in turn with 10% aqueous citric acid and with water. The organic solution was dried over anhydrous magnesium sulphate and evaporated. The residue was purified by column chromatography on silica using a 49:1 mixture of methylene chloride and methanol as eluent. There was thus obtained tert-butyl[5-(5-bromopyridin-3-yl)-1-methyl-1H-pyrazol-3-yl]carbamate (1.53 g); ¹H NMR Spectrum: (DMSOd₆) 1.47 (s, 9H), 3.76 (s, 3H), 6.55 (s, 1H), 8.28 (t, 1H), 8.74 (d, 1H), 8.77 (d, 1H), 9.61 (br s, 1H).

A mixture of the material so obtained, trifluoroacetic acid (10 ml) and methylene chloride (20 ml) was stirred at ambient temperature for 1 hour. The mixture was evaporated. The residue was basified by the addition of a saturated aqueous sodium bicarbonate solution (40 ml) and extracted with 10% methanol in methylene chloride. The organic solution was dried over anhydrous magnesium sulphate and evaporated. The residue was purified by column chromatography on silica using 2-5% methanol in methylene chloride as eluent. There was thus obtained 5-(5-bromopyridin-3-yl)-1-methyl-1H-pyrazol-3-amine (1.14 g); ¹H NMR Spectrum: (DMSOd₆) 3.64 (s, 3H), 4.69 (s, 2H), 5.74 (s, 1H), 8.19 (t, 1H), 8.69 (d, 1H), 8.73 (d, 1H); Mass Spectrum: M+H⁺ 253.

A mixture of the material so obtained and acetic anhydride (5 ml) was stirred and heated to 50° C. for 30 minutes. The mixture was cooled to ambient temperature and diluted with diethyl ether (40 ml). The resultant solid was isolated, washed with diethyl ether and dried to give N-[5-(5-bromopyridin-3-yl)-1-methyl-1H-pyrazol-3-yl]acetamide (1.04 g); ¹H NMR Spectrum: (DMSOd₆) 2.02 (s, 3H), 3.8 (s, 3H), 6.76 (s, 1H), 8.29 (t, 1H), 8.75 (d, 1H), 8.78 (d, 1H), 10.46 (s, 1H); Mass Spectrum: M+H⁺ 295.

Under an atmosphere of nitrogen, a mixture of a portion (0.819 g) of the material so obtained, diphenylmethanimine (benzophenone imine; 0.604 g), sodium tert-butoxide (0.801 g), tris(dibenzylideneacetone)dipalladium(0) (0.077 g), racemic 2,2′-bis(diphenylphosphino)-1,1′-binaphthyl (0.078 g) and 1,4-dioxane (15 ml) was stirred and heated to 100° C. for 40 minutes. The mixture was cooled to ambient temperature, diluted with 10% methanol in methylene chloride (40 ml) and filtered. The filtrate was concentrated and the residue was purified by column chromatography on silica using 7% methanol in methylene chloride as eluent. There was thus obtained N-{5-[5-(diphenylmethyleneamino)pyridin-3-yl]-1-methyl-1H-pyrazol-3-yl}acetamide (0.975 g); Mass Spectrum: M+H⁺ 396.

A mixture of the material so obtained, 2N aqueous hydrochloric acid (2 ml) and THF (16 ml) was stirred at ambient temperature for 1 hour. The resultant mixture was basified by the addition of a saturated aqueous sodium bicarbonate solution and extracted with 10% methanol in methylene chloride. The organic solution was dried over anhydrous magnesium sulphate and evaporated. The residue was purified by column chromatography on silica using increasingly polar mixtures of methylene chloride and methanol (containing 0-2% methanolic ammonia) as eluent. The material so obtained was triturated under diethyl ether. The resultant solid was isolated, washed with diethyl ether and dried to give N-[5-(5-aminopyridin-3-yl)-1-methyl-1H-pyrazol-3-yl]acetamide (0.492 g); ¹H NMR Spectrum: (DMSOd₆) 2.01 (s, 3H), 3.75 (s, 3H), 5.5 (s, 2H), 6.59 (s, 1H), 7.03 (t, 1H), 7.88 (d, 1H), 7.99 (d, 1H), 10.4 (s, 1H); Mass Spectrum: M+H⁺ 232.

### EXAMPLE 2

### N-[5-(5-benzenesulphonamidopyridin-3-yl)-1-methyl-1H-pyrazol-3-yl]acetamide

Benzenesulphonyl chloride (0.111 ml) was added dropwise to a stirred suspension of N-[5-(5-aminopyridin-3-yl)-1-methyl-1H-pyrazol-3-yl]acetamide (0.1 g) in pyridine (2 ml) and the resultant mixture was stirred at ambient temperature for 40 minutes. The mixture was diluted with methylene chloride (20 ml) and evaporated. The residue was purified by column chromatography on silica using 5-7% methanol in methylene chloride as eluent. The material so obtained was triturated under diethyl ether. The resultant solid was isolated, washed with diethyl ether and dried under vacuum. There was thus obtained the title compound (0.078 g); ¹H NMR Spectrum: (DMSOd₆) 2.02 (s, 3H), 3.67 (s, 3H), 6.62 (s, 1H), 7.53-7.69 (m, 4H), 7.80-7.84 (m, 2H), 8.35 (d, 1H), 8.44 (d, 1H), 10.45 (s, 1H), 10.76 (s, 1H); Mass Spectrum: M+H⁺ 372.

### EXAMPLE 3

### N-{5-[5-(5-chloro-1,3-dimethyl-1H-pyrazol-4-ylsulphonamido)pyridin-3-yl]-1-methyl-1H-pyrazol-3-yl}acetamide

5-Chloro-1,3-dimethyl-1H-pyrazol-4-ylsulphonyl chloride (0.157 ml) was added at ambient temperature to a stirred mixture of N-[5-(5-aminopyridin-3-yl)-1-methyl-1H-pyrazol-3-yl]acetamide (0.1 g) and pyridine (2 ml). The resultant mixture was stirred at ambient temperature for 15 minutes. The mixture was heated to 50° C. for 20 minutes. The mixture was cooled to ambient temperature and pyrrolidine (0.5 ml) was added. The resultant mixture was stirred at ambient temperature for 30 minutes. The mixture was evaporated and the residue was purified by column chromatography on silica using 5-7% methanol in methylene chloride as eluent. The material so obtained was triturated under diethyl ether. The resultant solid was isolated, washed with diethyl ether and dried under vacuum. There was thus obtained the title compound (0.16 g); ¹H NMR Spectrum: (DMSOd₆) 2.02 (s, 3H), 2.28 (s, 3H), 3.73 (s, 3H), 3.75 (s, 3H), 6.64 (s, 1H), 7.56 (t, 1H), 8.37 (d, 1H), 8.49 (d, 1H), 10.46 (s, 1H), 10.86 (s, 1H); Mass Spectrum: M+H⁺ 424 and 426.

The 5-chloro-1,3-dimethyl-1H-pyrazol-4-ylsulphonyl chloride used as a starting material is commercially available and is also described in J. Chem. Research Synopses 1986, 388.

### EXAMPLE 4

### N-{5-[5-(2,4-dimethylthiazol-5-ylsulphonamido)pyridin-3-yl]-1-methyl-1H-pyrazol-3-yl}acetamide

2,4-Dimethylthiazol-5-ylsulphonyl chloride (0.153 ml) was added at ambient temperature to a stirred mixture of N-[5-(5-aminopyridin-3-yl)-1-methyl-1H-pyrazol-3-yl]acetamide (0.1 g) and pyridine (2 ml). The resultant mixture was stirred at ambient temperature for 15 minutes. The mixture was heated to 50° C. for 20 minutes. The mixture was cooled to ambient temperature and pyrrolidine (0.5 ml) was added. The resultant mixture was stirred at ambient temperature for 30 minutes. The mixture was evaporated and the residue was purified by column chromatography on silica using 5-7% methanol in methylene chloride as eluent. The material so obtained was triturated under diethyl ether. The resultant solid was isolated, washed with diethyl ether and dried under vacuum. There was thus obtained the title compound (0.102 g); ¹H NMR Spectrum: (DMSOd₆) 2.02 (s, 3H), 2.41 (s, 3H), 2.62 (s, 3H), 3.73 (s, 3H), 6.66 (s, 1H), 7.62 (t, 1H), 8.39 (d, 1H), 8.55 (d, 1H), 10.47 (s, 1H), 11.06 (s, 1H); Mass Spectrum: M+H⁺ 407.

The 2,4-dimethylthiazol-5-ylsulphonyl chloride used as a starting material is commercially available and is also described in J. Het. Chem., 1981, 18, 997. The material may also be prepared as follows:—

Chlorosulphonic acid (20 ml) was cooled to 15° C. in an ice/methanol bath. 2,4-Dimethylthiazole (11.32 g) was added dropwise over 45 minutes, with the evolution of hydrogen chloride gas during the addition. The mixture so obtained was heated to 140-150° C. for 16 hours. The resultant mixture was cooled to 110-120° C. and finely powdered phosphorus pentachloride (41.6 g) was added in small portions, with the evolution of further hydrogen chloride gas during the addition. The mixture so obtained was heated to 120° C. for 1 hour. The mixture was cooled to ambient temperature and poured slowly into a vigorously stirred mixture of ice (200 g) and water (200 ml). The mixture so obtained was stirred for 30 minutes. The mixture was extracted with methylene chloride. The organic extract was dried over magnesium sulphate and purified by chromatography on silica using increasingly polar mixtures of isohexane and diethyl ether as eluent. There was thus obtained 2,4-dimethylthiazol-5-ylsulphonyl chloride as a yellow oil (18.4 g); ¹H NMR Spectrum: (CDCl₃) 2.76 (3H, s), 2.77 (3H, s).

### EXAMPLE 5

### N-{5-[5-(1,3,5-trimethyl-1H-pyrazol-4-ylmethylamino)pyridin-3-yl]-1-methyl-1H-pyrazol-3-yl}acetamide

A mixture of N-[5-(5-aminopyridin-3-yl)-1-methyl-1H-pyrazol-3-yl]acetamide (0.1 g), 1,3,5-trimethyl-1H-pyrazole-4-carbaldehyde (0.072 g), polymer-bound sodium cyanoborohydride (MP-cyanoborohydride from Argonaut Technologies Inc.; 2.04 mmol per g; 0.3 g), glacial acetic acid (0.075 ml) and methanol (3 ml) was stirred at ambient temperature for 8 hours. The mixture was filtered and the filtrate was evaporated. The residue was purified by column chromatography on silica using 10% methanol in methylene chloride as eluent. The material so obtained was triturated under diethyl ether and the resultant solid was isolated, washed with diethyl ether and dried under vacuum. There was thus obtained the title compound (0.071 g); ¹H NMR Spectrum: (DMSOd₆) 2.02 (s, 3H), 2.11 (s, 3H), 2.21 (s, 3H), 3.64 (s, 3H), 3.75 (s, 3H), 4.0 (d, 2H), 6.02 (t, 1H), 6.61 (s, 1H), 7.02 (t, 1H), 7.89 (d, 1H), 8.06 (d, 1H), 10.4 (s, 1H); Mass Spectrum: M+H⁺ 354.

The 1,3,5-trimethyl-1H-pyrazole-4-carbaldehyde used as a starting material is commercially available.

1. A pyrazole derivative of the Formula I 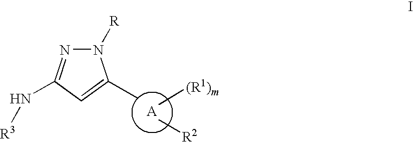
wherein the R group is hydrogen, (1-6C)alkyl or (3-8C)cycloalkyl,
or the R group is a (1-3C)alkyl group that bears a substituent selected from cyano, hydroxy, amino, (1-6C)alkoxy, (1-6C)alkylthio, (1-6C)alkylsulphinyl, (1-6C)alkylsulphonyl, (1-6C)alkylamino, di-[(1-6C)alkyl]amino, phenoxy, benzyloxy, phenylthio, phenylsulphinyl and phenylsulphonyl,
and wherein any phenyl group within a R group optionally bears 1, 2 or 3 substituents, which may be the same or different, selected from halogeno, trifluoromethyl, cyano, hydroxy, amino, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino and di-[(1-6C)alkyl]amino;
Ring A is a 2-pyridyl, 3-pyridyl, 5-pyrimidinyl, 2-pyrazinyl or 4-pyridazinyl group;
m is 0, 1 or 2;
each R¹ group that is present, which may be the same or different, is selected from halogeno, trifluoromethyl, cyano, hydroxy, amino, (1-8C)alkyl, (2-8C)alkenyl, (2-8C)alkynyl and (1-6C)alkoxy;
the R² group is selected from halogeno, trifluoromethyl, cyano, hydroxy, amino, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoyloxy, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
—X²-Q²
wherein X² is a direct bond or is selected from O, S, SO, SO₂, N(R⁵), CO, CH(OR⁵), CON(R⁵), N(R⁵)CO, N(R⁵)CON(R⁵), SO₂N(R⁵), N(R⁵)SO₂, C(R⁵)₂O, C(R⁵)₂S and C(R⁵)₂N(R⁵), wherein each R⁵ group is hydrogen, (1-8C)alkyl or (2-6C)alkanoyl, and Q² is aryl, aryl-(1-6C)alkyl, aryloxy-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any CH, CH₂ or CH₃ group within a R² group optionally bears on each said CH, CH₂ or CH₃ group one or more halogeno or (1-8C)alkyl substituents and/or a substituent selected from hydroxy, mercapto, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyloxy, (2-6C)alkanoylamino and N-(1-6C)alkyl-(2-6C)alkanoylamino, or from a group of the formula:
—X³-Q³
wherein X³ is a direct bond or is selected from O, S, SO, SO₂, N(R⁶) and CO, wherein R⁶ is hydrogen or (1-8C)alkyl, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, heteroaryl or heterocyclyl group within a R² group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (2-8C)alkenyl, (2-8C)alkynyl, (1-6C)alkoxy, (2-6C)alkenyloxy, (2-6C)alkynyloxy, (1-6C)alkylthio, (1-6C)alkylsulphinyl, (1-6C)alkylsulphonyl, (1-6C)alkylamino, di-[(1-6C)alkyl]amino, (1-6C)alkoxycarbonyl, (2-6C)alkanoyl, (2-6C)alkanoyloxy, N-(1-6C)alkylcarbamoyl, N,N-di-[(1-6C)alkyl]carbamoyl, (2-6C)alkanoylamino, N-(1-6C)alkyl-(2-6C)alkanoylamino, N-(1-6C)alkylsulphamoyl, N,N-di-[(1-6C)alkyl]sulphamoyl, (1-6C)alkanesulphonylamino and N-(1-6C)alkyl-(1-6C)alkanesulphonylamino, or from a group of the formula:
—X⁴—R⁷
wherein X⁴ is a direct bond or is selected from O and N(R⁸), wherein R⁸ is hydrogen or (1-8C)alkyl, and R⁷ is halogeno-(1-6C)alkyl, hydroxy-(1-6C)alkyl, (1-6C)alkoxy-(1-6C)alkyl, (1-6C)alkylthio-(1-6C)alkyl, (1-6C)alkylsulphinyl-(1-6C)alkyl, (1-6C)alkylsulphonyl-(1-6C)alkyl, cyano-(1-6C)alkyl, amino-(1-6C)alkyl, (1-6C)alkylamino-(1-6C)alkyl, di-[(1-6C)alkyl]amino-(1-6C)alkyl, (2-6C)alkanoylamino-(1-6C)alkyl or N-(1-6C)alkyl-(2-6C)alkanoylamino-(1-6C)alkyl, or from a group of the formula:
—X⁵-Q⁴
wherein X⁵ is a direct bond or is selected from O, CO and N(R⁹), wherein R⁹ is hydrogen or (1-8C)alkyl, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl,
and wherein any heterocyclyl group within the R² group optionally bears 1 or 2 oxo or thioxo substituents; and
the R³ group is selected from formyl, carboxy, carbamoyl, (1-6C)alkoxycarbonyl, N-(1-6C)alkylcarbamoyl, N,N-di-[(1-6C)alkyl]carbamoyl, (2-6C)alkanoyl, (3-8C)cycloalkylcarbonyl, N-(1-6C)alkylsulphamoyl and N,N-di-[(1-6C)alkyl]sulphamoyl, or from a group of the formula:
Q⁵-X⁶
wherein X⁶ is selected from CO, N(R¹⁰)CO and N(R¹⁰)SO₂, wherein R¹⁰ is hydrogen or (1-8C)alkyl, and Q⁵ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any CH, CH₂ or CH₃ group within a R³ group optionally bears on each said CH, CH₂ or CH₃ group one or more halogeno or (1-8C)alkyl substituents and/or a substituent selected from hydroxy, amino, cyano, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyloxy, (2-6C)alkanoylamino and N-(1-6C)alkyl-(2-6C)alkanoylamino,
and wherein any aryl, (3-8C)cycloalkyl, heteroaryl or heterocyclyl group within a R³ group optionally bears 1, 2 or 3 substituents, which may be the same or different, selected from halogeno, trifluoromethyl, cyano, nitro, trifluoromethoxy, hydroxy, amino, (1-8C)alkyl, (2-8C)alkenyl, (2-8C)alkynyl, (1-6C)alkoxy, (1-6C)alkylthio, (1-6C)alkylsulphinyl, (1-6C)alkylsulphonyl, (1-6C)alkylamino, di-[(1-6C)alkyl]amino, (2-6C)alkanoyl, (2-6C)alkanoyloxy, (2-6C)alkanoylamino and N-(1-6C)alkyl-(2-6C)alkanoylamino,
and wherein any heterocyclyl group within a R³ group optionally bears 1 or 2 oxo or thioxo substituents;
or a pharmaceutically-acceptable salt thereof.

2. A pyrazole derivative of the Formula II 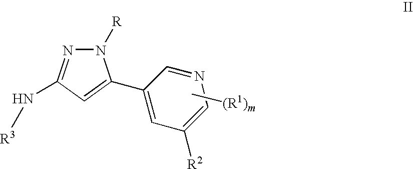
wherein each of R, m, R¹, R² and R³ has any of the meanings defined in claim 1;
or a pharmaceutically-acceptable salt thereof.

3. A pyrazole derivative of the Formula II 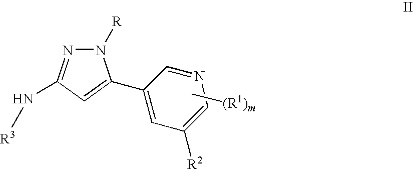
wherein R¹ is a (1-6C)alkylamino group or a group of the formula:
—NH-Q²
wherein Q² has any of the meanings defined in claim 1;
and each of R, m, R¹ and R³ has any of the meanings defined in claim 1;
or a pharmaceutically-acceptable salt thereof.

4. A pyrazole derivative of the Formula II 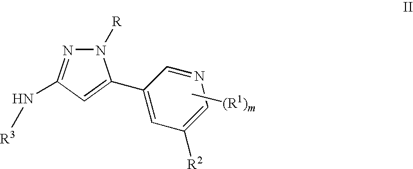
wherein R² is a (1-6C)alkanesulphonylamino group or a group of the formula:
—NHSO₂-Q²
wherein Q² has any of the meanings defined in claim 1;
and each of R, m, R¹ and R³ has any of the meanings defined in claim 1;
or a pharmaceutically-acceptable salt thereof.

5. A pyrazole derivative of the Formula I according to claim 1
wherein R is (1-3C)alkyl;
and each of m, R¹, R² and R³ has any of the meanings defined in claim 1;
or a pharmaceutically-acceptable salt thereof.

6. A pyrazole derivative of the Formula I according to claim 1
wherein the R² group is selected from (1-6C)alkylamino, di-[(1-6C)alkyl]amino, (2-6C)alkanoylamino and (1-6C)alkanesulphonylamino, or from a group of the formula:
—X²-Q²
wherein X² is selected from NH, NHCO and NHSO₂, and Q² is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any CH₂ or CH₃ group within a R² group optionally bears on each said CH₂ or CH₃ group one or more halogeno or (1-8C)alkyl substituents and/or a substituent selected from hydroxy, (1-6C)alkoxy, (1-6C)alkylamino and di-[(1-6C)alkyl]amino, or from a group of the formula:
—X³-Q³
wherein X³ is a direct bond or is selected from O and NH, and Q³ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, heteroaryl or heterocyclyl group within a R² group optionally bears 1, 2 or 3 substituents, which may be the same or different, selected from halogeno, trifluoromethyl, cyano, hydroxy, amino, nitro, trifluoromethoxy, carboxy, carbamoyl, (1-8C)alkyl, (1-6C)alkoxy, (1-6C)alkylsulphonyl, (1-6C)alkylamino, di-[(1-6C)alkyl]amino, (1-6C)alkoxycarbonyl, N-(1-6C)alkylcarbamoyl, N,N-di-[(1-6C)alkyl]carbamoyl, (2-6C)alkanoyl, (2-6C)alkanoylamino and N-(1-6C)alkyl-(2-6C)alkanoylamino, or from a group of the formula:
—X⁴—R⁷
wherein X⁴ is O and R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:
—X⁵-Q⁴
wherein X⁵ is a direct bond or O, and Q⁴ is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, hydroxy, (1-8C)alkyl, (1-6C)alkoxy, (1-6C)alkylthio, (1-6C)alkylsulphinyl, (1-6C)alkylsulphonyl and (2-6C)alkanoyl;
and each of R, m, R¹ and R³ has any of the meanings defined in claim 1;
or a pharmaceutically-acceptable salt thereof.

7. A pyrazole derivative of the Formula I according to claim 1
wherein R² is a (1-6C)alkylamino group or a group of the formula:
—NH-Q²
wherein Q² is aryl-(1-6C)alkyl, (3-8C)cycloalkyl-(1-6C)alkyl, heteroaryl-(1-6C)alkyl or heterocyclyl-(1-6C)alkyl,
and wherein any aryl, (3-8C)cycloalkyl, heteroaryl or heterocyclyl group within a R² group optionally bears 1 or 2 substituents, which may be the same or different, selected from halogeno, trifluoromethyl, cyano, hydroxy, amino, (1-8C)alkyl, (1-6C)alkoxy, (1-6C)alkylsulphonyl, (1-6C)alkylamino, di-[(1-6C)alkyl]amino, (2-6C)alkanoyl and (2-6C)alkanoylamino, or from a group of the formula:
—O—R⁷
wherein R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:
—X⁵-Q⁴
wherein X⁵ is a direct bond or O, and Q⁴ is aryl, aryl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, (1-8C)alkyl, (1-6C)alkoxy, (1-6C)alkylsulphonyl and (2-6C)alkanoyl;
and each of R, m, R¹ and R³ has any of the meanings defined in claim 1;
or a pharmaceutically-acceptable salt thereof.

8. A pyrazole derivative of the Formula I according to claim 1
wherein R² is a (1-6C)alkanesulphonylamino group or a group of the formula:
—NHSO₂-Q²
wherein Q² is aryl, aryl-(1-6C)alkyl, (3-8C)cycloalkyl, (3-8C)cycloalkyl-(1-6C)alkyl, heteroaryl, heteroaryl-(1-6C)alkyl, heterocyclyl or heterocyclyl-(1-6C)alkyl,
and wherein any aryl, (3-8C)cycloalkyl, heteroaryl or heterocyclyl group within a R² group optionally bears 1 or 2 substituents, which may be the same or different, selected from halogeno, trifluoromethyl, cyano, hydroxy, amino, carboxy, (1-8C)alkyl, (1-6C)alkoxy, (1-6C)alkylsulphonyl, (1-6C)alkylamino, di-[(1-6C)alkyl]amino, (1-6C)alkoxycarbonyl, (2-6C)alkanoyl, (2-6C)alkanoylamino and N-(1-6C)alkyl-(2-6C)alkanoylamino, or from a group of the formula:
—O—R⁷
wherein R⁷ is hydroxy-(1-6C)alkyl, (1-6C)alkoxy-(1-6C)alkyl, cyano-(1-6C)alkyl, (1-6C)alkylamino-(1-6C)alkyl and di-[(1-6C)alkyl]amino-(1-6C)alkyl, or from a group of the formula:
—X⁵-Q⁴
wherein X⁵ is a direct bond or O, and Q⁴ is aryl, aryl-(1-6C)alkyl, heteroaryl, heteroaryl-(1-6C)alkyl, heterocyclyl or heterocyclyl-(1-6C)alkyl, and the Q⁴ group optionally bears 1 or 2 substituents, which may be the same or different, selected from halogeno, cyano, (1-8C)alkyl, (1-6C)alkoxy, (1-6C)alkylsulphonyl and (2-6C)alkanoyl;
and each of R, m, R¹ and R³ has any of the meanings defined in claim 1;
or a pharmaceutically-acceptable salt thereof.

9. A pyrazole derivative of the Formula II 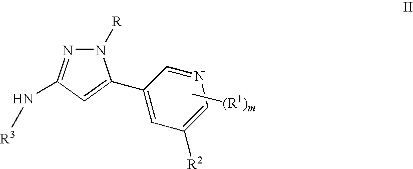
wherein:—
R is methyl, ethyl or propyl;
m is 0 or m is 1 and the R¹ group is selected from fluoro, chloro, bromo, trifluoromethyl, cyano, methyl, ethyl, methoxy and ethoxy;
R² is methanesulphonylamino, ethanesulphonylamino, propanesulphonylamino, 2,2-difluoroethanesulphonylamino, 2,2,2-trifluoroethanesulphonylamino, 2-chloroethanesulphonylamino, 3-chloropropanesulphonylamino, 2-hydroxyethanesulphonylamino, 3-hydroxypropanesulphonylamino, 3-methylaminopropanesulphonylamino, 3-dimethylaminopropanesulphonylamino, 3-ethylaminopropanesulphonylamino, 3-diethylaminopropanesulphonylamino, 3-cyclopentylaminopropanesulphonylamino, 3-cyclohexylaminopropanesulphonylamino, 3-(cyclopentylmethylamino)propanesulphonylamino, 3-(cyclohexylmethylamino)propanesulphonylamino, 3-morpholinopropanesulphonylamino, 3-pyrrolidin-1-ylpropanesulphonylamino, 3-piperidinopropanesulphonylamino, 3-piperazin-1-ylpropanesulphonylamino, 3-(4-methylpiperazin-1-yl)propanesulphonylamino or 3-benzylaminopropanesulphonylamino, or R² is a group of the formula:
—N(R⁵)SO₂-Q²
wherein R⁵ is hydrogen, methyl, ethyl or acetyl, and Q² is phenyl, benzyl, cyclopropyl, cyclobutyl, cyclopentyl, cyclohexyl, cyclopropylmethyl, cyclobutylmethyl, pyrrolyl, furyl, thienyl, imidazolyl, pyrazolyl, oxazolyl, isoxazolyl, thiazolyl, isothiazolyl, oxadiazolyl, thiadiazolyl, triazolyl, pyridyl, pyrazinyl, pyrimidinyl or pyridazinyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, methyl, ethyl, methoxy, ethoxy, methylthio and methylsulphonyl; and
R³ is carbamoyl, N-methylcarbamoyl, N-ethylcarbamoyl, N-propylcarbamoyl, N-isopropylcarbamoyl, N-(2-hydroxyethyl)carbamoyl, N-(3-hydroxypropyl)carbamoyl, N-(2-methoxyethyl)carbamoyl, N-(3-methoxypropyl)carbamoyl, acetyl, propionyl, benzoyl, furylcarbonyl, thienylcarbonyl, pyridylcarbonyl, benzylcarbonyl, N-phenylcarbamoyl, N-benzylcarbamoyl, N-cyclopropylcarbamoyl, N-(furylmethyl)carbamoyl, N-(thienylmethyl)carbamoyl and N-(isoxazolylmethyl)carbamoyl, and each of the eleven last named substituents optionally bears 1 or 2 substituents, which may be the same or different, selected from fluoro, chloro, bromo, cyano, hydroxy, methyl, ethyl, methoxy and ethoxy;
or a pharmaceutically-acceptable salt thereof.

10. A pyrazole derivative of the Formula II according to claim 9 wherein:—
R is methyl or ethyl;
m is 0 or m is 1 and the R¹ group is selected from fluoro, chloro, bromo, methyl, ethyl and methoxy;
R² is methanesulphonylamino, ethanesulphonylamino or propanesulphonylamino, or a group of the formula:
—NHSO₂-Q²
wherein Q² is phenyl, benzyl, cyclopropyl, cyclopropylmethyl, 2-furyl, 3-furyl, 2-thienyl, 3-thienyl, 4-imidazolyl, 4-pyrazolyl, 5-oxazolyl, 4-isoxazolyl, 5-thiazolyl, 4-isothiazolyl or 3-pyridyl, each of which optionally bears 1, 2 or 3 substituents, which may be the same or different, selected from fluoro, chloro, bromo, trifluoromethyl, cyano, hydroxy, amino, methyl, ethyl, methoxy, ethoxy, methylsulphonyl, methylamino, dimethylamino, acetyl and acetamido;
and R³ is acetyl;
or a pharmaceutically-acceptable salt thereof.

11. A pyrazole derivative of the Formula II according to claim 9 wherein:—
R is methyl;
m is 0 or m is 1 and the R¹ group is selected from chloro and methyl;
R² is methanesulphonylamino, or a group of the formula:
—NHSO₂-Q²
wherein Q² is phenyl, 5-thiazolyl or 4-pyrazolyl, each of which optionally bears 1, 2 or 3 substituents, which may be the same or different, selected from fluoro, chloro and methyl; and
R³ is acetyl;
or a pharmaceutically-acceptable salt thereof.

12. A pyrazole derivative of the Formula II according to claim 9 wherein —
R is methyl, ethyl or propyl;
m is 0 or m is 1 and the R¹ group is selected from fluoro, chloro, bromo, trifluoromethyl, cyano, methyl, ethyl, methoxy and ethoxy;
R¹ is amino, methylamino, ethylamino, propylamino, dimethylamino, diethylamino, 2-hydroxyethylamino, 3-hydroxypropylamino, 3-methylaminopropylamino, 3-dimethylaminopropylamino, 3-ethylaminopropylamino or 3-diethylaminopropylamino, or R² is a group of the formula:
—N(R⁵)-Q²
wherein R⁵ is hydrogen, methyl or ethyl, and Q² is benzyl, pyrrolylmethyl, furylmethyl, thienylmethyl, imidazolylmethyl, pyrazolylmethyl, oxazolylmethyl, isoxazolylmethyl, thiazolylmethyl, isothiazolylmethyl, oxadiazolylmethyl, thiadiazolylmethyl, triazolylmethyl, pyridylmethyl, pyrazinylmethyl, pyrimidinylmethyl or pyridazinylmethyl, each of which optionally bears 1, 2 or 3 substituents, which may be the same or different, selected from fluoro, chloro, bromo, trifluoromethyl, cyano, hydroxy, amino, nitro, trifluoromethoxy, carboxy, carbamoyl, methyl, ethyl, methoxy, ethoxy, methylsulphonyl, methylamino, dimethylamino, methoxycarbonyl, acetyl, 2,2,2-trifluoroacetyl, acetamido, N-methylacetamido, propionamido, N-methylpropionamido, 2-hydroxyethoxy, 3-hydroxypropoxy, 2-cyanoethoxy, 3-cyanopropoxy, 2-methylaminoethoxy, 3-methylaminopropoxy, 2-dimethylaminoethoxy, 3-dimethylaminopropoxy, pyrrolidin-1-yl, piperidino, morpholino, piperazin-1-yl, 4-methylpiperazin-1-yl, phenyl, benzyl, pyridyl, pyrimidinyl, pyrazinyl, phenoxy and pyridyloxy, and each of the seven last named substituents optionally bears 1 or 2 substituents, which may be the same or different, selected from fluoro, chloro, bromo, cyano, hydroxy, methyl, ethyl, methoxy, ethoxy, methylthio and methylsulphonyl; and
R³ is carbamoyl, N-methylcarbamoyl, N-ethylcarbamoyl, N-propylcarbamoyl, N-isopropylcarbamoyl, N-(2-hydroxyethyl)carbamoyl, N-(3-hydroxypropyl)carbamoyl, N-(2-methoxyethyl)carbamoyl, N-(3-methoxypropyl)carbamoyl, acetyl, propionyl, benzoyl, furylcarbonyl, thienylcarbonyl, pyridylcarbonyl, benzylcarbonyl, N-phenylcarbamoyl, N-benzylcarbamoyl, N-cyclopropylcarbamoyl, N-(furylmethyl)carbamoyl, N-(thienylmethyl)carbamoyl and N-(isoxazolylmethyl)carbamoyl, and each of the eleven last named substituents optionally bears 1 or 2 substituents, which may be the same or different, selected from fluoro, chloro, bromo, cyano, hydroxy, methyl, ethyl, methoxy and ethoxy;
or a pharmaceutically-acceptable salt thereof.

13. A pyrazole derivative of the Formula II according to claim 9 wherein: —
R is methyl or ethyl;
m is 0 or m is 1 and the R¹ group is selected from fluoro, chloro, bromo, methyl, ethyl and methoxy;
R² is a group of the formula:
—NH-Q²
wherein Q² is benzyl, 2-pyrrolylmethyl, 3-pyrrolylmethyl, 2-furylmethyl, 3-furylmethyl, 2-thienylmethyl, 3-thienylmethyl, 4-imidazolylmethyl, 4-pyrazolylmethyl, 5-oxazolylmethyl, 4-isoxazolylmethyl, 5-thiazolylmethyl, 4-isothiazolylmethyl, 1,2,3-triazol-4-ylmethyl and 3-pyridylmethyl, each of which optionally bears 1, 2 or 3 substituents, which may be the same or different, selected from fluoro, chloro, bromo, trifluoromethyl, cyano, hydroxy, amino, methyl, ethyl, methoxy, ethoxy, methylsulphonyl, methylamino, dimethylamino, acetyl and acetamido; and
R³ is acetyl;
or a pharmaceutically-acceptable salt thereof.

14. A pyrazole derivative of the Formula II according to claim 9 wherein —
R is methyl;
m is 0 or m is 1 and the R¹ group is selected from chloro and methyl;
R² is a group of the formula:
—NH-Q²
wherein Q² is 4-pyrazolylmethyl which optionally bears 1, 2 or 3 substituents, which may be the same or different, selected from fluoro, chloro and methyl; and
R³ is acetyl;
or a pharmaceutically-acceptable salt thereof.

15. A pharmaceutical composition which comprises a pyrazole derivative of the Formula I, or a pharmaceutically-acceptable salt thereof, according to claim 1 in association with a pharmaceutically-acceptable diluent or carrier.
